(19) United States
(12) Patent Application Publication
(10) Pub. No.: US 2016/0220759 A1
(43) Pub. Date: Aug. 4, 2016

(54) Injection Device for Selective Fixed or Variable Dosing

(71) Applicant: NOVO NORDISK A/S, Bagsværd (DK)

(72) Inventors: Christian Peter Enggaard, Vejby (DK); Ebbe Kiilerich, Copenhagen NV (DK); Michael Frank Petersen, Solroed Strand (DK); Mads Groenlund Andersen, Copenhagen NV (DK)

(21) Appl. No.: 15/021,174

(22) PCT Filed: Sep. 16, 2014
(86) PCT No.: PCT/EP2014/069695

§ 371 Date: Mar. 10, 2016

Related U.S. Application Data
(60) Provisional application No. 61/878,803, filed on Sep. 17, 2013.

(30) Foreign Application Priority Data
Sep. 16, 2013 (EP) 13184598.4
Dec. 20, 2013 (EP) 13199032.7
Jul. 25, 2014 (EP) 14178575.8

(51) Int. Cl. A61M 5/315

(52) U.S. Cl. CPC A61M 5/31541; A61M 5/31551; A61M 2005/3126

(57) ABSTRACT

A drug delivery device (1; 100; 200) comprising a housing (2; 102; 201, 202), a dose setting mechanism operable to set a dose to be delivered from a variable volume reservoir, and a dose delivery structure (60; 160; 260) activatable to expel a set dose. The dose setting mechanism comprises a dose indicating structure (40; 140; 240) for indicating a size of the set dose. The dose indicating structure (40; 140; 240) is coupled with the dose delivery structure (60; 160; 260) during expelling of the set dose and moved relative to the housing (2; 102; 201, 202) to a zero dose indicating position. The zero dose indicating position is a position which is fixed with respect to the housing (2; 102; 201, 202). The dose setting mechanism further comprises a dose setting structure (20; 120; 220) movable in a dose preparing direction relative to the housing (2; 102; 201, 202) to a dose prepared position to set a dose of a first size. The dose prepared position is a position along the dose preparing direction which is fixed with respect to the housing (2; 102; 201, 202). The dose setting structure (20; 120; 220) and the dose indicating structure (40; 140; 240) are coupled and configured to undergo first correlated displacements relative to the housing (2; 102; 201, 202) during movement of the dose indicating structure (40; 140; 240) to the zero dose indicating position and further coupled and configured to undergo second correlated displacements relative to the housing (2; 102; 201, 202) during movement of the dose setting structure (20; 20; 220) to the dose prepared position, where the first correlated displacements and the second correlated displacements are mutually reverse. Further, when the dose setting structure (20; 120; 220) is in the dose prepared position the dose indicating structure (40; 140; 240) is selectively displaceable relative to the housing (2; 102; 201, 202) while the dose setting structure (20; 120; 220) remains stationary in the dose preparing direction to allow adjustment of the dose of the first size and thereby setting of a dose of a second size.

### FIELD OF THE INVENTION

The present invention relates to drug delivery devices, e.g. to power assisted drug delivery devices such as automatic injection devices. In particular, the invention relates to dose setting mechanisms for such delivery devices.

### BACKGROUND OF THE INVENTION

Within some therapy areas the tendency of a patient to adhere to the prescribed therapy is dependent on the simplicity of the specific treatment regimen. For example, many people with type 2 diabetes are diagnosed with the disease at a relatively high age where they are less prone to accept a treatment that intervenes too much with their normal way of living. Most of these people do not like constantly being reminded of their disease and, as a consequence, they do not want to be entangled in complex treatment patterns or waste time on learning to operate cumbersome delivery systems.

Basically, people with diabetes need to minimise their glucose excursions. Insulin is a well-known glucose lowering agent which must be administered parenterally to be effective in the body. The presently most common way of administering insulin is by subcutaneous injections. Such injections have traditionally been performed using a vial and a syringe, but in recent years so-called injection devices, or injection pens, have gained more and more attention in the marketplace. Many people find these injection devices easier to handle and generally more convenient than the vial and syringe solution. For example, because an injection pen carries a prefilled drug container, or is adapted to receive a prefilled drug container, the user is not required to carry out a separate filling procedure before each injection.

Some prior art injection devices suitable for self-injection are adapted to deliver multiple settable doses of drug. The user can set a desired dose by operating a dose setting mechanism and subsequently inject the set dose by operating an injection mechanism. In this case the dose is variable, i.e. the user must set a dose which is suitable in the specific situation each time a dose is to be injected. An example of such an injection device is found in U.S. Pat. No. 5,226,896 (Eli Lilly and Company).

Other prior art injection devices are adapted to repeatedly deliver a fixed dose of drug. These devices are typically designed to simplify the administration procedure for people following a therapeutic treatment regimen which involves intermittently injecting the same amount of a drug, and they accordingly offer quick and easy preparation of the fixed dose. An example of such a fixed dose injection device is found in WO 2005/039676 (Eli Lilly and Company).

While the above mentioned types of injection devices are quite suitable for delivery of various kinds of drugs according to various treatment plans they do possess obvious drawbacks. For example, in regard to a variable dose injection device each and every dose delivery procedure requires the user to pay particular attention to the dose display during dose setting in order to assure that the correct dose is set before an injection is commenced. In particular when it comes to all mechanical devices striving to be as small and handy as possible the surface useable for displaying the set dose is rather limited and visually impaired users often have trouble reading the physically small dose indicating numerals. In regard to a fixed dose injection device even the most constant treatment regimens may need a dose adjustment at some point, e.g. during a dose titration period, in which case the fixed dose injection device becomes useless and another injection device adapted to deliver a dose of a different size is needed.

WO 2009/098299 (Novo Nordisk A/S) discloses a fixed dose injection device which offers an opportunity to change a predetermined fixed dose to a new fixed dose level by rotation of an adjustment ring. This allows a user to change from one predetermined fixed dose to another predetermined fixed dose in a simple manner, useful for example during dose titration. However, the user can only choose between a few predetermined fixed doses selected by the manufacturer, and the feature is therefore only suitable for a limited group of people.

In view of the above there is a need for an even more flexible drug delivery device which offers a greater possibility to follow a specific dose regimen, whether it be fixed, varied, or a combination thereof, while still being simple to use and easy to carry about during the day.

### SUMMARY OF THE INVENTION

It is an object of the invention to eliminate or reduce at least one drawback of the prior art, or to provide a useful alternative thereto.

In particular, it is an object of the invention to provide a drug delivery device which can function selectively as a fixed dose device or as a variable dose device, according to the user's needs or desires.

It is a further object of the invention to provide such a drug delivery device which is safe to use and simple to handle.

It is an even further object of the invention to provide a drug delivery device of the above mentioned kind which requires relatively few constructional components, thereby minimising its manufacturing costs.

In the disclosure of the present invention, aspects and embodiments will be described which will address one or more of the above objects and/or which will address objects apparent from the following text.

A drug delivery device embodying the principles of the present invention comprises a housing, a dose setting mechanism for setting a dose to be delivered from a substance reservoir when the substance reservoir is coupled with the housing, and a dose delivery mechanism, where the dose setting mechanism comprises a dose setting structure operable to set an initial dose and a dose indicating structure, where the dose setting structure and the dose indicating structure are coupled to undergo correlated displacements relative to the housing during setting of the initial dose, respectively during dose delivery, where the correlated displacements during setting of the initial dose are opposite the correlated displacements during dose delivery, where the dose setting structure is moved to a dose prepared position to set the initial dose, the dose prepared position being fixed relative to the housing, and the dose indicating structure is moved to a dose stop position during dose delivery, the dose stop position being fixed relative to the housing, and where the dose indicating structure is selectively displaceable relative to the housing when the dose setting structure is in the dose prepared position to thereby allow adjustment of the initial dose and setting of a final dose.

Thus, in one aspect of the invention a drug delivery device is provided comprising:
a housing,
a dose setting mechanism operable to set a dose to be delivered from a substance reservoir when the substance reservoir is coupled with the housing, and
a dose delivery structure activatable during a dose expelling procedure to cause expelling of a set dose,
   wherein the dose setting mechanism comprises:
a dose indicating structure for indicating a size of the set dose, the dose indicating structure being coupled with the dose delivery structure during the dose expelling procedure and moved relative to the housing to a zero dose indicating position, and
a dose setting structure adapted to be moved in a dose preparing direction relative to the housing to a dose prepared position to set a dose of a first size,
   wherein the zero dose indicating position is fixed with respect to the housing,
   wherein the dose prepared position is a position along the dose preparing direction which is fixed with respect to the housing,
   wherein the dose setting structure and the dose indicating structure are coupled and configured to undergo first correlated displacements relative to the housing during movement of the dose indicating structure to the zero dose indicating position and further coupled and configured to undergo second correlated displacements relative to the housing during movement of the dose setting structure to the dose prepared position, the first correlated displacements and the second correlated displacements being mutually reverse, and
   wherein when the dose setting structure is in the dose prepared position the dose indicating structure is selectively displaceable relative to the housing while the dose setting structure remains stationary in the dose preparing direction to allow adjustment of the dose of the first size and thereby setting of a dose of a second size.

In another aspect of the invention a drug delivery device is provided comprising:
a housing,
a dose setting structure, and
a dose indicating structure, the dose indicating structure being configured to move from a first dose set position to a zero dose indicating position in response to an operation of a dose activation means to cause a dose delivery structure to deliver a corresponding metered dose from a substance reservoir, the zero dose indicating position being fixed with respect to the housing, and the dose setting structure being configured to move from a dose prepared position to an end-of-dose position in response to a movement of the dose indicating structure from the first dose set position to the zero dose indicating position,
   wherein the dose setting structure is further movable in a dose preparing direction from the end-of-dose position to the dose prepared position, and the dose indicating structure is further configured to move from the zero dose indicating position to the first dose set position in response to a movement of the dose setting structure from the end-of-dose position to the dose prepared position, and when the dose setting structure is in the dose prepared position the dose indicating structure is selectively repositionable to a second dose set position while the dose setting structure remains stationary in the dose preparing direction, and
   wherein the dose prepared position is a position along the dose preparing direction which is fixed with respect to the housing.

In a further aspect of the invention a drug delivery device is provided comprising:
a housing,
a dose setting structure movable in a dose preparing direction relative to the housing to a dose prepared position to define a prepared dose to be delivered from a substance reservoir, the dose prepared position being a position along the dose preparing direction which is fixed with respect to the housing,
a dose indicating structure selectively manipulable when the dose setting structure is in the dose prepared position to adjust the prepared dose and define an adjusted dose to be delivered from the substance reservoir, the dose indicating structure being subsequently movable a first distance or a second distance relative to the housing to a zero dose indicating position in accordance with a delivery of either the prepared dose or the adjusted dose, the zero dose indicating position being fixed with respect to the housing, and
a dose delivery structure responsive to an operation of a dose activation means to be coupled with the dose indicating structure and cause movement of the dose indicating structure to the zero dose indicating position to thereby effect the dose delivery, and to be decoupled from the dose indicating structure thereupon,
   wherein when the dose indicating structure is being moved to the zero dose indicating position during dose delivery the dose setting structure and the dose indicating structure undergo first correlated displacements relative to the housing, and when the dose setting structure is subsequently being moved to the dose prepared position the dose setting structure and the dose indicating structure undergo second correlated displacements relative to the housing, the first correlated displacements and the second correlated displacements being mutually reverse, and
   wherein when the dose setting structure is in the dose prepared position displacement of the dose indicating structure relative to the housing is allowed while the dose setting structure remains stationary in the dose preparing direction with respect to the housing.

The substance reservoir, which may be a variable volume reservoir, e.g. comprising a selectively openable and closable drug outlet and a movable wall (such as a conventional cartridge type reservoir comprising a self-sealing septum and a slidable piston), may be nonreleasably coupled with the housing or may be adapted to be coupled with the housing, e.g. via a reservoir support structure configured to hold at least a portion of the substance reservoir, before use of the drug delivery device. Regardless of which, when the substance reservoir is coupled with the housing the drug outlet defines an outlet end of the drug delivery device.

The zero dose indicating position is the position which the dose indicating structure takes up when no dose has been set, e.g. following a complete delivery of a dose and before the automatic dose setting resulting from a movement of the dose setting structure to the dose prepared position. In the zero dose indicating position the dose indicating structure signals to the surroundings that no dose is set, e.g. by virtue of being in a certain visually inspectable position relative to the housing. In this certain position the dose indicating structure may e.g. convey a “0” or a like unequivocal symbol.

In accordance with the above aspects of the invention a dose setting procedure for the inventive drug delivery device comprises a dose preparation and a selective dose adjustment (at least after the very first dose delivery, as the drug delivery device may be offered by the manufacturer in a pre-use state where the dose setting structure is already in the dose prepared position, while the dose indicating structure is in the zero dose indicating position). Following a first dose delivery the dose preparation is executed by movement of the dose setting structure to the dose prepared position, which is essentially a dose ready position indicating that a set dose is ready for delivery. Movement of the dose setting structure from a first end-of-dose position to the dose prepared position causes a movement of the dose indicating structure which is exactly the reverse of the movement that it underwent during the previous movement of the dose setting structure from the dose prepared position to the first end-of-dose position in connection with the first dose delivery. Thereby, a dose is prepared which corresponds to the dose that was last delivered. This enables a user to use the device as a fixed dose delivery device for a selective number of dose deliveries and thereby avoid having to repeatedly carry out a dose setting procedure that requires scrutiny of small dose indicating numerals. If the dose needs to be changed at a certain point in time the user can easily adjust the prepared dose and set a new dose that corresponds exactly to the dose needed by simple manipulation of the dose indicating structure after dose preparation. Further, the automatic setting of a dose equaling the one that was last delivered provides for an inexpensive mechanical memory in the drug delivery device which allows the user to easily verify the size of the latest administered dose. Such verification is attractive, especially for people who self-administer medication on a regular basis because the administration act itself tends to become a matter of routine, i.e. the procedure is in risk of being carried out without sufficient attention, entailing an increased likelihood of e.g. the dose being confused with other recent administrations.

The dose preparing direction may be an axial direction, such as e.g. a longitudinal direction being parallel to a longitudinal axis of the housing, a rotational direction, or a combination of an axial and a rotational direction. In other words, the dose setting structure may be movable to the dose prepared position by translation, rotation, or helical motion, relative to the housing.

It is noted that the phrase “the dose setting structure remains stationary in the dose preparing direction with respect to the housing” designates that the dose setting structure neither moves in the dose preparing direction, nor in the direction opposite to the dose preparing direction (relative to the housing).

The drug delivery device as defined by the above aspects of the invention can be realised by use of relatively few constructional components and is therefore relatively inexpensive to produce. Further, because the zero dose indicating position and the dose prepared position are well-defined and unchangeable with respect to the housing the dose preparation solution is both accurate and reproducible, as the prepared dose (i.e. the dose of the first size) only depends on the relative position of the dose setting structure and the dose indicating structure at the onset of a dose administration. Also, any accidental, or deliberate, user provoked change of one or both of the zero dose indicating position and the dose prepared position with a resulting compromise of the mechanical memory function is prevented.

The dose setting structure and the dose indicating structure may be arranged concentrically along the longitudinal axis, and the dose indicating structure may surround at least a portion of the dose setting structure. This enables the provision of a slender dose setting mechanism which may serve to reduce the overall size of the drug delivery device.

Further, the dose delivery structure and the dose setting structure may be arranged concentrically, and the dose setting structure may surround at least a portion of the dose delivery structure. Thereby, the drug delivery device may be realised as a pen-type device having a generally circular cylindrical configuration. Such devices may be preferred by some, as they are of a particularly slender design.

The dose preparation (i.e. the setting of the initial dose) may be effected automatically in response to a predetermined user action. The user action may comprise directly contacting the dose setting structure and moving the dose setting structure to a stop, or operating a dose arming structure to abut the dose setting structure, or an intermediate element operatively coupled with the dose setting structure, and move the dose setting structure, or the intermediate element, to a stop. The stop may e.g. be a position defined by an abutment of the dose setting structure or the intermediate element with a wall, or a position defined by an extent of possible movement of a portion of the dose arming structure relative to the housing.

In particular embodiments of the invention, the drug delivery device further comprises a cap receiving portion adapted to receive and releasably retain a cap in a position where the cap covers a distal most portion of the drug delivery device, e.g. at least an end portion of the substance reservoir, and the predetermined user action comprises mounting the cap onto the cap receiving portion. In some of these embodiments the cap comprises the dose arming structure, while in others the dose arming structure forms part of the drug delivery device, and the cap is configured to interact with the dose arming structure during mounting onto the cap receiving portion. Either way, a very simple drug delivery device is provided because the cap can be used as a protective cover for the distal portion of the drug delivery device while also being useable to automatically prepare the drug delivery device for delivery of a dose of drug. The dose preparation step is thus in practice carried out unnoticed by the user, as it is integrated into a normal use pattern of the drug delivery device, which already includes a mounting and dismounting of the cap on/from the cap receiving portion between two dose deliveries.

When the dose setting structure is in the dose prepared position a user may selectively manipulate the dose indicating structure to adjust the prepared dose, i.e. the user has the option to set a final dose which differs from the last dose delivered, if she/he so desires. Such manipulation may be enabled automatically in response to the dose setting structure reaching the dose prepared position, or manually subsequent to the dose setting structure reaching the dose prepared position, e.g. by release of a lock.

The dose indicating structure may comprise dose related indicia usable in a display of the set dose, and the housing may comprise a window through which the dose related indicia are successively viewable, e.g. one indicium at a time. Thereby, the position of the dose indicating structure relative to the housing will be correlated with, and indicative of, the size of the actual set dose. Particularly, the dose indicating structure may be or comprise a scale drum, an odometer, or the like.

The drug delivery device may further comprise a user operable dose adjustment structure, e.g. in the form of a dose dial, for displacing the dose indicating structure relative to the housing. In that case the dose adjustment structure is operable to displace the dose indicating structure only when the dose setting structure is in the dose prepared position. This provides for an additional safety measure, as it is thus not possible to manually reposition the dose indicating structure with respect to the housing during a dose delivery procedure (i.e. an operation of the dose activation means leading to an expelling of a set dose from the device), e.g. during a temporary pausing of the dose expelling, and thereby introduce uncertainty as to the dose actually being delivered.

The dose adjustment structure may be configured to be decoupled from the dose indicating structure in response to the dose setting structure being moved away from the dose prepared position, and further to be coupled with the dose indicating structure in response to the dose setting structure being brought to the dose prepared position.

Alternatively, or additionally, the dose adjustment structure may be configured to be rendered inoperable in response to the dose setting structure being moved away from the dose prepared position, and further to be rendered operable in response to the dose setting structure being brought to the dose prepared position.

In some embodiments of the invention the dose adjustment structure comprises a dose dial and the dose indicating structure comprises a scale drum, and the dose dial is rotationally coupled with the scale drum when the dose setting structure is in the dose prepared position and rotationally decoupled from the scale drum when the dose setting structure is moved away from the dose prepared position.

In a particular embodiment of the invention the dose setting structure comprises a non-self-locking thread on a first exterior surface portion and a longitudinal groove on a second exterior surface portion bordering on the first exterior surface portion, and the drug delivery device further comprises a rotator serving as an intermediate connecting piece for the dose setting structure and the dose indicating structure. The rotator comprises an exterior longitudinal track, which longitudinal track is adapted for engagement with a protrusion on an interior surface of the dose indicating structure so as to provide a rotational interlocking connection between the rotator and the dose indicating structure. The rotator further comprises an interior projection adapted for engagement with the non-self-locking thread, when the dose setting structure is in the dose prepared position, and for rotational interlocking connection with the longitudinal groove when the dose setting structure is away from the dose prepared position, thereby allowing for a helical displacement of the dose indicating structure in response to a translational displacement of the dose setting structure during dose delivery as well as during dose preparation and for a helical displacement of the dose indicating structure in response to a rotational displacement of the dose setting structure, when the dose setting structure is in the dose prepared position.

The dose delivery structure may comprise a piston rod, or a pressure plate, or a like structure capable of applying a force to the substance reservoir. In particular embodiments of the invention the dose delivery structure comprises a piston rod configured to cause displacement of a piston in a cartridge. The dose delivery structure may further comprise a drive member for actuating the piston rod (or the like structure) in response to an operation of the dose activation means.

The dose activation means may comprise a user operable dose activation button shiftable between a passive position and an activated position. A shifting of the dose activation button to the activated position may cause movement of the dose indicating structure towards the zero dose indicating position. Further, the dose delivery structure may be configured to be coupled with the dose indicating structure in response to the dose activation button being shifted to the activated position, and to be decoupled from the dose indicating structure in response to the dose activation button being shifted to the passive position.

The drug delivery device may be power assisted, e.g. spring assisted, in which case it further comprises a spring means capable of storing and releasing energy for actuation of the dose delivery structure. The spring means may be pre-tensioned and dimensioned to effect an emptying of the substance reservoir without being re-tensioned, or it may be adapted to be tensioned in connection with e.g. a dose setting activity. The spring means may particularly comprise a torque inducing spring, such as e.g. a spiral spring, or a force inducing spring, such as e.g. a compression spring.

The spring means may be retained when the dose activation button is in the passive position and released when the dose activation button is in the activated position. Particularly, the spring means may be configured to be released in response to the dose activation button being shifted from the passive position to the activated position and to be detained in response to the dose activation button being shifted from the activated position to the passive position during expelling of the set dose. This enables a pausing of an ongoing dose administration by a shift of the dose activation button to the passive position.

The dose activation button may be biased towards the passive position, e.g. by a return spring, whereby a pausing of an ongoing dose administration may be obtained simply by the user terminating the force being applied to the dose activation button.

The dose activation button and/or the dose dial may be arranged with respect to the housing (e.g. in the distal half of the housing) so as to become covered by the cap when the cap is mounted onto the cap receiving portion. This will automatically prevent any undesired operation of the dose activation button and/or the dose dial when the drug delivery device is in a non-use state, i.e. without requiring a conscious action by the user.

In the present context the term “mutually reverse”, as used in connection with the correlated displacements of the dose setting structure and the dose indicating structure, designates that the individual displacements of the dose setting structure and the dose indicating structure relative to the housing when the dose indicating structure is being moved to the zero dose indicating position during dose delivery, respectively when the dose setting structure is subsequently being moved to the dose prepared position are identical in size but opposite in direction.

Further, in the present context the term “proximal” refers to a portion, position or direction opposite or away from the outlet end of the drug delivery device, whereas “distal”, conversely, refers to a portion, position or direction close to or towards the outlet end of the drug delivery device.

In the present specification, reference to a certain aspect or a certain embodiment (e.g. “an aspect”, “a first aspect”, “one embodiment”, “an exemplary embodiment”, or the like) signifies that a particular feature, structure, or characteristic described in connection with the respective aspect or embodiment is included in, or inherent of, at least that one aspect or embodiment of the invention, but not necessarily in/of all aspects or embodiments of the invention. It is emphasized, however, that any combination of the various features, structures and/or characteristics described in relation to the invention is encompassed by the invention unless expressly stated herein or clearly contradicted by context.

The use of any and all examples, or exemplary language (e.g., such as, etc.), in the text is intended to merely illuminate the invention and does not pose a limitation on the scope of the same, unless otherwise claimed. Further, no language or wording in the specification should be construed as indicating any non-claimed element as essential to the practice of the invention.

### BRIEF DESCRIPTION OF THE DRAWINGS

In the following the invention will be further described with references to the drawings, wherein

FIGS. 1a-1h illustrate the movement pattern of certain elements of a drug delivery device in an exemplary embodiment of the invention,

FIG. 2 is a longitudinal section view of a drug delivery device according to another embodiment of the invention,

FIG. 3a is a perspective view of a portion of the drug delivery device,

FIG. 3b shows an enlargement of a section of the drug delivery device shown in FIG. 3a,

FIG. 4 is a perspective view of a scale drum used in the drug delivery device,

FIG. 5 is a perspective view of a dose defining rod used in the drug delivery device,

FIGS. 6a and 6b are different view detailing a rotator used in the drug delivery device, and

FIGS. 7-12 are longitudinal section views of the drug delivery device in different states during use.

FIG. 13 is a perspective view of a portion of an injection device according to a further embodiment of the invention,

FIG. 14 is a perspective view detailing elements of a drive mechanism in the injection device of FIG. 13,

FIGS. 15-19 are perspective views of the portion of the injection device in different states during use,

FIG. 20 is an exploded view of a drug delivery device according to yet a further embodiment of the invention,

FIG. 21 is an exploded view of a nut assembly used in the drug delivery device of FIG. 20,

FIG. 22 is a longitudinal section view of the drug delivery device,

FIG. 23 is a close-up section view of a proximal portion of the drug delivery device as delimited by the area Q in FIG. 22,

FIG. 24 is a perspective longitudinal section view of the nut assembly in a locked state corresponding to the state of the drug delivery device shown in FIG. 23,

FIG. 25 is a close-up section view of a proximal portion of the drug delivery device showing the nut assembly in an unlocked state,

FIG. 26 is a close-up section view of a proximal portion of the drug delivery device showing the nut assembly during drug delivery, and

FIG. 27 is a perspective longitudinal section view of the nut assembly in a position corresponding to the one shown in FIG. 26.

In the figures like structures are mainly identified by like reference numerals.

### DESCRIPTION OF EXEMPLARY EMBODIMENTS

When in the following relative expressions, such as “upwards” and “downwards”, are used, these refer to the appended figures and not necessarily to an actual situation of use. The shown figures are schematic representations for which reason the configuration of the different structures as well as their relative dimensions are intended to serve illustrative purposes only.

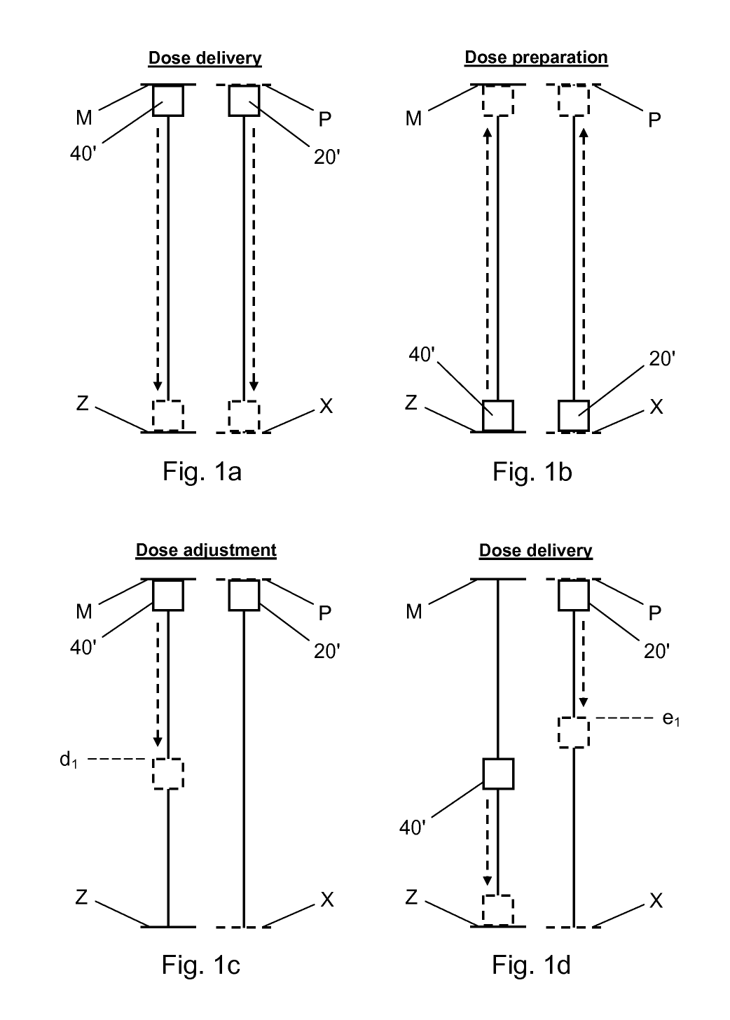

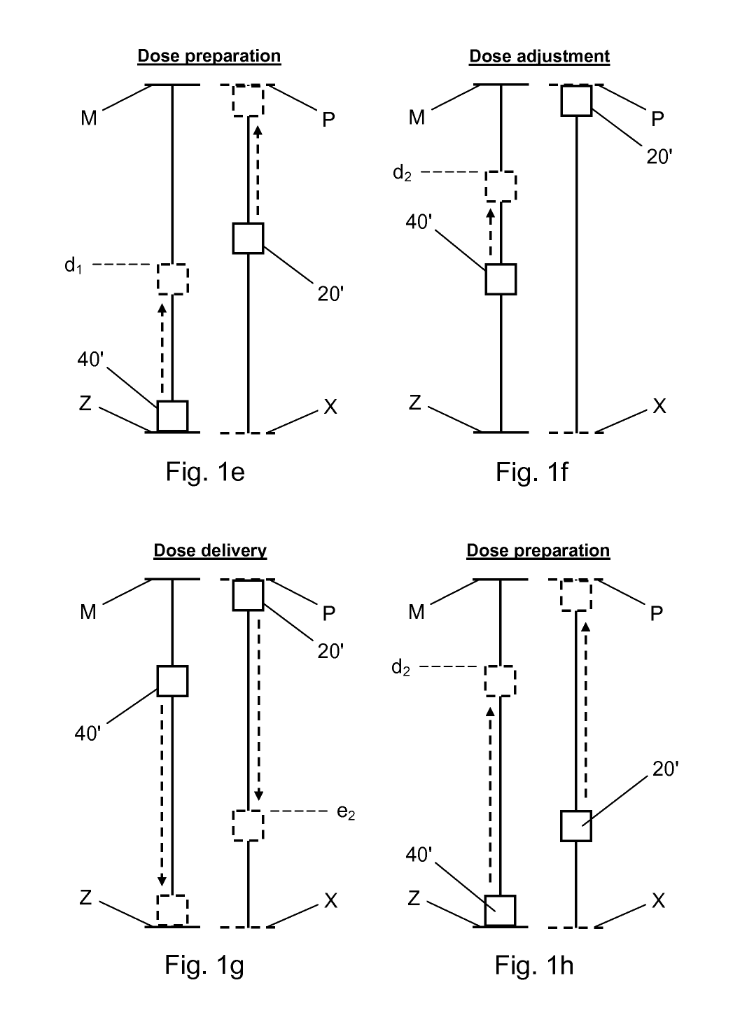

FIG. 1 is a simplified schematic representation of the principles underlying the present invention. FIGS. 1a through 1h illustrate the movement pattern of certain reciprocative drug delivery device elements during dose setting and dose delivery, respectively. The respective movements are to be understood as displacements relative to a base structure of the drug delivery device (not shown in FIG. 1), such as e.g. a drug delivery device housing. In the interest of clarity, the movements are shown as purely axial movements along straight lines, but it is noted that they could just as well be purely rotational movements between angularly offset points of reference, or any combination of axial and rotational movements.

As described previously the dose setting procedure to be carried out by a user of a drug delivery device according to the present invention comprises a dose preparation step and an optional dose adjustment step. The dose preparation step will automatically set a dose corresponding to the dose that was last ejected, which will be clear from the below, while the optional dose adjustment step allows a user to change the automatically set dose to a new dose, which then becomes a final set dose. Depending on whether the user has carried out the dose adjustment step or not the final set dose ejected from the drug delivery device in response to an execution of the dose delivery procedure is either the automatically set dose or the new dose. In case the user has made use of the opportunity to change the automatically set dose and the dose ejected thereby differs from the previous dose ejected the next dose preparation step will automatically set a dose corresponding to the new dose. Thereby, if the treatment plan prescribes that the same dose is administered each time the user simply abstains from performing the dose adjustment step and benefits from an effortless automatic dose setting, but she/he has the option to change the dose at any time should the treatment so require.

In FIG. 1a a dose preparation element 20′ is shown in solid lines at a line P that defines a dose prepared position for the dose preparation element 20′. When the dose preparation element 20′ is at the line P the drug delivery device is in a “DOSE PREPARED” state in which it is ready to deliver a dose from a drug reservoir (not shown). The dose prepared position of the dose preparation element 20′ is an axial position which is fixed with respect to the aforementioned base structure of the drug delivery device.

Further, a dose defining element 40′ is shown in solid lines in a first extreme position in which it abuts a stop surface M that defines a maximum dose set position for the dose defining element 40′. When the dose defining element 40′ abuts the stop surface M the maximum settable dose for the drug delivery device is set. Thus, when the dose preparation element 20′ and the dose defining element 40′ are in the shown positions the drug delivery device is ready to deliver the maximum settable dose.

FIG. 1a illustrates the movement pattern of the dose preparation element 20′ and the dose defining element 40′ during dose delivery. The stippled arrows indicate that during dose delivery the dose preparation element 20′ and the dose defining element 40′ are coupled and undergo correlated movements, whereby the dose defining element 40′ moves from the maximum dose set position to a second extreme position in which it abuts a stop surface Z that defines a zero dose indicating position for the dose defining element 40′, and the dose preparation element 20′ moves from the dose prepared position to a position indicated by a line X that defines a maximum dose delivered position for the dose preparation element 20′.

A dose delivery mechanism (not shown) is coupled with the dose defining element 40′ during movement of the dose defining element 40′ from the maximum dose set position to the zero dose indicating position to cause the maximum settable dose to be expelled from the drug reservoir.

When the dose defining element 40′ abuts the stop surface Z the drug delivery device is in a “DOSE DELIVERED” state in which a set dose has been delivered and no further dose delivery can take place until the dose setting procedure has been carried out. The stop surface Z is fixed with respect to the base structure of the drug delivery device. As will be clear from the following, a movement of the dose defining element 40′ to the zero dose indicating position is, however, only accompanied by a movement of the dose preparation element 20′ to the maximum dose delivered position when the maximum settable dose is being delivered.

FIG. 1b illustrates the movement pattern of the dose preparation element 20′ and the dose defining element 40′ during the subsequent dose preparation. The stippled arrows again indicate that the dose preparation element 20′ and the dose defining element 40′ are coupled and undergo correlated movements. Thus, when the dose preparation element 20′ moves back to the dose prepared position, e.g. as a result of the user performing a simple predetermined action, the dose defining element 40′ moves back accordingly to the maximum dose set position. Thereby, the maximum settable dose has automatically been prepared for delivery. During the dose preparation the dose delivery mechanism is decoupled from the dose defining element 40′. It is noted that the direction in which the dose preparation element 20′ moves towards the dose prepared position is referred to as the dose preparing direction.

FIG. 1c illustrates the optional dose adjustment which the user may choose to make use of to alter the prepared dose and set a different final dose to be delivered. When, or after, the dose preparation element 20′ reaches the dose prepared position, as shown with solid lines, the dose preparation element 20′ and the dose defining element 40′ become decoupled with respect to movements in the dose preparing direction, allowing the user to reposition the dose defining element 40′ relative to the dose preparation element 20′ and the base structure of the drug delivery device, while the dose preparation element 20′ remains stationary, at least in the dose preparing direction. This repositioning is indicated by the stippled arrow. In FIG. 1c the dose defining element 40′ is moved to a position d₁, which is a dose set position corresponding to a particular dose, “Dose 1”, smaller than the maximum settable dose. “Dose 1” is thus the final set dose which will be delivered in response to the next execution of the dose delivery procedure. In case the user chooses not to reposition the dose defining element 40′ relative to the dose preparation element 20′ then the prepared maximum settable dose will be delivered in response to the next execution of the dose delivery procedure.

FIG. 1d illustrates the movement pattern of the dose preparation element 20′ and the dose defining element 40′ during delivery of “Dose 1”. The dose preparation element 20′ and the dose defining element 40′ are once again coupled and undergo correlated movements, whereby the dose defining element 40′ is brought from position d₁ to the zero dose indicating position and the dose preparation element 20′ is brought from the dose prepared position to a position e₁ between the dose prepared position and the maximum dose delivered position. The position e₁ is thus an end-of-dose position for the dose preparation element 20′ corresponding to the delivery of “Dose 1”.

FIG. 1e illustrates the movement pattern of the dose preparation element 20′ and the dose defining element 40′ during the subsequent dose preparation. Here, because of the operative coupling between the dose preparation element 20′ and the dose defining element 40′ the reverse movement of the dose preparation element 20′ from position e₁ to the dose prepared position, e.g. resulting from a predetermined user action similar or identical to the one previously performed, causes a movement of the dose defining element 40′ back to position d₁. The drug delivery device is thus brought into a “DOSE PREPARED” state in which it is automatically ready to deliver “Dose 1” once more.

FIG. 1f illustrates another dose adjustment, following the preparation of “Dose 1”. Because the dose preparation element 20′ has reached the dose prepared position repositioning of the dose defining element 40′ relative to the dose preparation element 20′ is possible and the dose defining element 40′ is moved to a position d₂, which corresponds to another particular dose, “Dose 2”, smaller than the maximum settable dose but larger than “Dose 1”. “Dose 2” is now the final set dose which will be delivered in response to the next execution of the dose delivery procedure.

FIG. 1g illustrates the movement pattern of the dose preparation element 20′ and the dose defining element 40′ during delivery of “Dose 2”. The two elements are once again coupled and undergo correlated movements, bringing the dose defining element 40′ from position d₂ to the zero dose indicating position and the dose preparation element 20′ from the dose prepared position to a position e₂ between position e₁ and the maximum dose delivered position. Position e₂ is thus an end-of-dose position for the dose preparation element 20′ corresponding to the delivery of “Dose 2”.

In FIG. 1h, illustrating the subsequent dose preparation, the dose preparation element 20′ is moved to the dose prepared position, e.g. in response to a predetermined user action, causing the dose defining element 40′ to move back to position d₂ due to the operative coupling with the dose preparation element 20′. Thus, the predetermined user action has this time caused “Dose 2” to be automatically prepared for delivery. Any subsequent execution of the dose delivery procedure without intermediate dose adjustment will therefore cause “Dose 2” to be delivered from the drug delivery device (as long as a dose of that particular size is available for delivery from the drug reservoir).

From the foregoing it is clear that every time a dose is being prepared the dose preparation element 20′ is brought to the dose prepared position, regardless of from which position it departs, and every time a dose is being delivered the dose defining element 40′ is brought to the zero dose indicating position, regardless of from which position it departs. Furthermore, the dose preparation element 20′ and the dose defining element 40′ are coupled and undergo mutually reverse correlated movements during dose preparation and dose delivery and are decoupled at least with respect to movements in the dose preparing direction when the drug delivery device is in the “DOSE PREPARED” state. It is therefore the position of the dose defining element 40′ relative to the base structure of the drug delivery device (e.g. the housing) in the “DOSE PREPARED” state that determines the size of the dose to be delivered. Each set dose matches a unique position of the dose defining element 40′ relative to the base structure of the drug delivery device, and each delivered dose matches a unique position of the dose preparation element 20′ relative to the base structure of the drug delivery device. This means that every time a dose has been delivered the dose preparation element 20′ holds a specific position relative to the base structure of the drug delivery device which depends on the size of the delivered dose, and because the dose preparation element 20′ and the dose defining element 40′ are coupled and undergo mutually reverse correlated movements during dose preparation and dose delivery every time a dose has been prepared the dose defining element 40′ has been brought back to the position from which it last departed. In other words, a movement of the dose preparation element 20′ to the dose prepared position will always cause an automatic preparation of a dose which equals the last dose delivered. This automatically prepared dose can then be selectively adjusted by the user to set a new final dose before the dose delivery procedure is carried out.

The above described solution is attractive from a user perspective because the device is simple to handle and offers automatic preparation of a dose corresponding to the one that has last been delivered in response to a simple predetermined user action, while it also offers selective manual adjustment of the prepared dose. It is also attractive from a manufacturing perspective because relatively few components are required to provide a highly accurate device offering a high degree of user convenience.

The respective stop surfaces M, Z in FIG. 1 make up axial stops for axial movements of the dose defining element 40′. It is noted, however, that within the scope of the invention these stops need not be axial, but may alternatively be rotational or transversal, or they may be a combination of axial, rotational and transversal.

Also, the various indicated correlated movements of the dose preparation element 20′ and the dose defining element 40′ in FIG. 1 may give the impression that the two elements move exactly the same distance in the same direction when a dose is prepared, respectively when a dose is delivered. This is, however, not necessarily the case. In different implementations of the inventive concept the dose preparation element 20′ and the dose defining element 40′ may in fact move different distances and/or in different directions during dose preparation and/or dose delivery, as will be clear from the below. The magnitude and direction of displacement of the respective elements are thus inessential to the practice of the invention, as long as the movements are correlated, i.e. as long as one particular movement of the one element (in the dose preparation/dose delivery phase) is always accompanied by one particular movement of the other element, and vice versa.

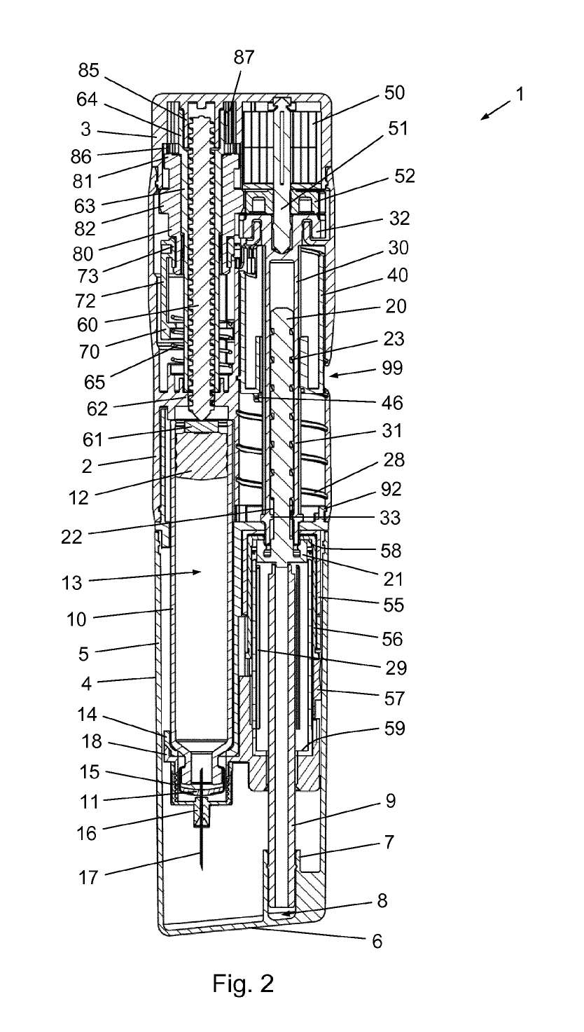

FIG. 2 displays a longitudinal section view of an injection device 1 according to an exemplary embodiment of the invention. The injection device 1, which is depicted in a pre-use state, has a base structure in the form of a housing 2 to which a drug cartridge 10 is attached. The cartridge 10 is axially fixed with respect to the housing 2 by means of a cartridge holder 14. The distal end portion of the cartridge 10 is closed by a penetrable self-sealing rubber septum 11 and the proximal end portion is sealed by a slidable rubber piston 12, the septum 11 and the piston 12 together defining a variable volume chamber 13 within the frame of the elongated cartridge wall.

A needle assembly, comprising an injection needle 17 fixedly retained in a needle hub 16, is mounted on a threaded needle interface 15 of a needle mount 18 such that the back end of the injection needle 17 transpierces the septum 11 and resides in the chamber 13.

A threaded piston rod 60 abuts the piston 12 via a piston rod foot 61 and is adapted to cause the piston 12 to move downwards in the cartridge 10 and force a volume of the substance in the chamber 13 out through the injection needle 17. The movement of the piston rod 60 is helical and guided by a nut 62 which is integrally formed with the housing 2. The piston rod 60 is in splined engagement with a piston rod guide 63 which is adapted to impart a rotary motion on the piston rod 60 during dose delivery. The piston rod guide 63 is an elongated cylindrical structure which surrounds a portion of the piston rod 60 and which has a proximal extension 64 that is fitted about an interior sleeve 85 of a spring housing 3 fixedly attached to the housing 2. The proximal extension 64 carries a couple of flexible arms (not visible) which interact with a ring of jagged teeth 87 arranged interiorly in the spring housing 3 to provide a ratchet mechanism ensuring a unidirectional rotation of the piston rod guide 63 relative to the housing 2.

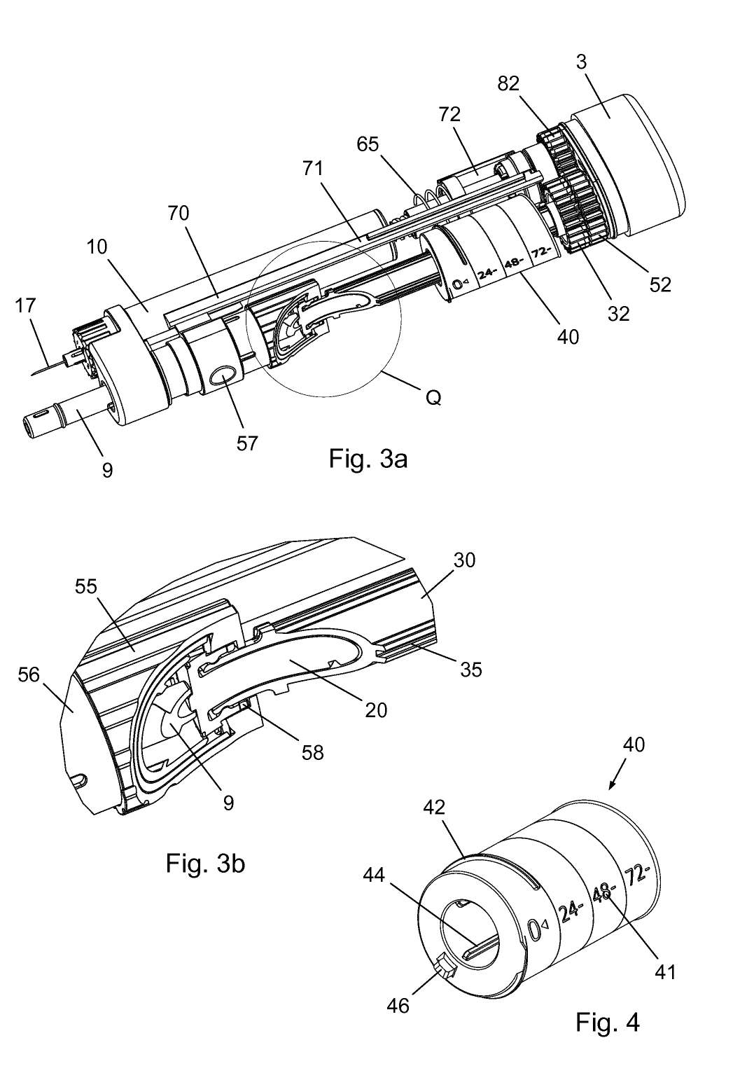

The piston rod guide 63 further has a toothed exterior belt portion (not visible) at its centre region which is adapted for engagement with an interior toothing (not visible) in an axially displaceable gear 80 during dose delivery, as will be explained below. The gear 80 has a proximal toothed rim portion 81 adapted for engagement with an interior circumferential toothing 86 in the spring housing 3, except from during dose delivery, and a central toothed rim portion 82. The gear 80 is axially displaceable by means of a transmission bar 70 which extends axially from the distal portion of the injection device 1, as is best seen in FIG. 3a, and which includes a coupling rod 72 with a catch portion 73 for engagement with a distal portion of the gear 80. The transmission bar 70 is biased towards the proximal end of the injection device 1 by a return spring 65, which means that the proximal toothed rim portion 81 is biased towards engagement with the circumferential toothing 86.

A pre-tensioned spiral spring 50 is arranged in the spring housing 3. The spring 50 comprises an outer spring end portion which is fixed to the spring housing 3 and an inner spring end portion which is fixed to a rotatable spring shaft 51. The spring shaft 51 extends axially downwards from the spring 50 and is rotationally locked to a cog wheel 52 which is in turn rotationally coupled with the central toothed rim portion 82 of the gear 80.

A rotator 30 having a proximal toothed head 32 is axially fixed to the housing 2 just distally of the cog wheel 52. The toothed head 32 is adapted to receive and engage with the central toothed rim portion 82 when the gear 80 is displaced distally against the bias of the return spring, as described below. The rotator 30 further comprises a hollow cylindrical sleeve 31 which accommodates an elongated dose preparation rod 20. The dose preparation rod 20 has an exterior helical groove 23 which terminates distally in a longitudinal groove 22. An interior protrusion 33 in the sleeve 31, which in FIG. 2 is positioned within the longitudinal groove 22, is adapted to travel at least a portion of the helical groove 23 during dose setting and dose delivery. The dose preparation rod 20 further comprises a coupling head 21 which in the shown state of the injection device 1 abuts an interior wall 58. The dose preparation rod 20 is able to move axially between the shown proximal position and a distal position in which the coupling head 21 abuts a radial wall 59.

The rotator 30 is further rotationally locked to a scale drum 40 which carries dose related indicia 41 (see FIG. 4) on an exterior surface and which is structured to undergo helical displacement along an interior thread 28 in the housing 2 when rotated by the rotator 30. In the shown pre-use state of the injection device 1 the scale drum 40 is in a zero dose indicating position relative to the housing 2 which indicates that no dose is set. Any particular position of the scale drum 40 relative to the housing 2 corresponds to a particular set dose, which is displayed through a window 99. From the position in FIG. 2 the scale drum 40 is displaceable helically downwards until a stud 46 on the distal end surface of the scale drum 40 meets a distal stud 92 in the housing 2, constituting a rotational stop for displacement of the scale drum 40 in the distal direction. This defines a bottom position of the scale drum 40 relative to the housing 2 and corresponds to a maximum settable dose being set.

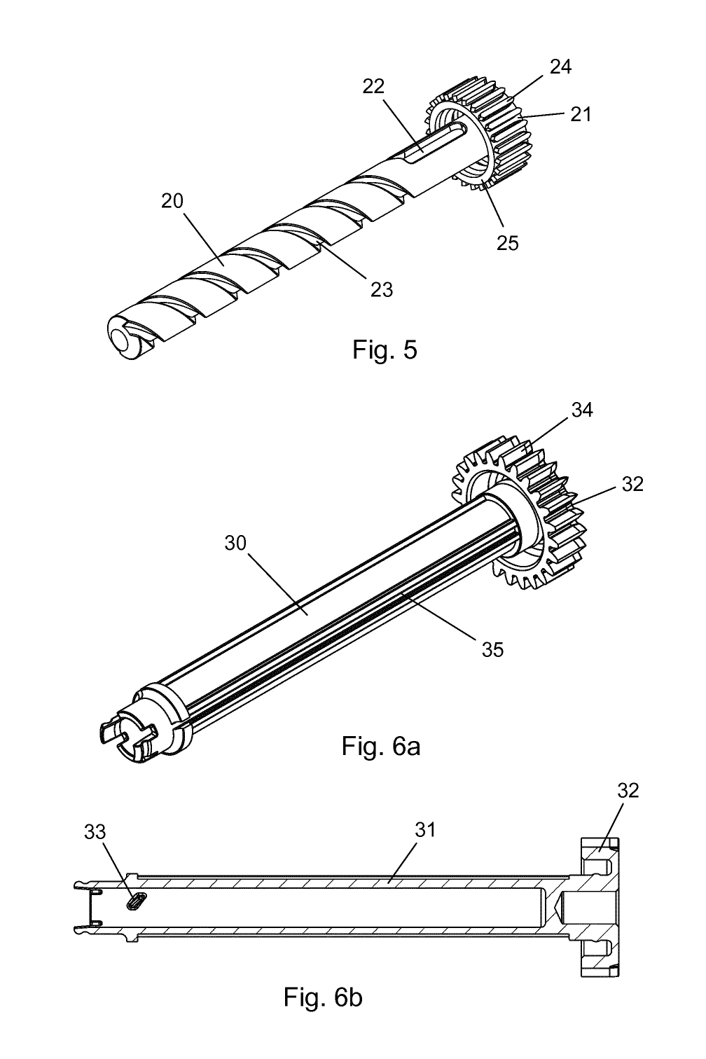

The coupling head 21 is provided with teeth 24 along its circumference (see FIG. 5) which in the shown position of the dose preparation rod 20 are in rotational engagement with an inner toothed portion of a dose dial 55. The dose dial 55 is adapted to be operated by a user to define a final dose that will be administered during dose delivery. During dose delivery the coupling head 21 is displaced distally whereby the teeth 24 are disengaged from the dose dial 55 and engaged with an interior spline 29 instead, preventing rotation of the dose preparation rod 20 relative to the housing 2.

An injection button 57 is arranged distally of the dose dial 55 and is axially slidable relative to the housing 2 between a proximal passive position and a distal activated position in which a dose delivery is initiated. The injection button 57 is axially locked to a carriage 56 which is configured to bring the dose preparation rod 20 to a dose start position in response to the injection button 57 being slid to the activated position.

In the pre-use state shown in FIG. 2 a cap 4 is mounted on the injection device 1, covering the distal portion of the cartridge 10, the needle assembly, the dose dial 55 and the injection button 57. The cap 4 comprises a cylindrical side wall 5 and an end wall 6. An interior sleeve 7 is formed on an inner surface of the end wall 6 and provides a cavity 8 for reception of an actuation rod 9. The actuation rod 9 is axially locked to the interior sleeve 7 and extends proximally through the radial wall 59 to the distal end surface of the coupling head 21. The cap 4 is received and releasably retained by a cap receiving portion of the housing 2.

FIG. 3a is a perspective view of the injection device 1 stripped of the housing 2 and the cap 4 to reveal that the transmission bar 70 is axially coupled at its distal end portion to the injection button 57. It is further seen that the transmission bar 70 comprises an axially extending leg 71. The leg 71 is axially coupled with the coupling rod 72 and the axial position of the gear 80 in the housing 2 is therefore determined by the axial position of the injection button 57 in the sense that when the injection button 57 is moved from the passive position to the activated position the transmission bar 70 and the gear 80 are urged distally against the bias of the return spring 65 and when the injection button 57 is released the return spring 65 moves the injection button 57 and thereby the transmission bar 70 and the gear 80 proximally backwards until the injection button 57 returns to the passive position.

FIG. 3b is an enlargement of the portion of the injection device 1 delimited by the area Q in FIG. 3a. The enlargement further details the various connections between the dose dial 55, the carriage 56, the dose preparation rod 20, and the rotator 30. In particular, the figure shows the rotational engagement between the dose dial 55 and the dose preparation rod 20, and the axial engagement between the carriage 56 and the dose preparation rod 20 via the interior wall 58. Also shown are axial splines 35 on the exterior of the rotator 30 used for rotationally locking the rotator 30 to the scale drum 40.

FIG. 4 is a perspective view of the scale drum 40, showing some of the dose related indicia 41 printed, embossed or otherwise applied to its peripheral surface. For the sake of clarity, only the numbers 0, 24, 48, and 72 are shown in this figure, but the scale drum 40 is preferably configured to also display set doses between these numbers, e.g. in single unit increments. A helical track segment 42 is arranged on the surface for engagement with the interior thread 28. Finally, the figure shows a longitudinal interior projection 44 adapted for reception in one of the splines 35. The scale drum 40 has three such projections distributed equidistantly along an interior circumference for reception in corresponding splines on the rotator 30 to ensure a stable rotational connection between the scale drum 40 and the rotator 30.

FIG. 5 is a perspective view of the dose preparation rod 20 which details that the helical groove 23 and the longitudinal groove 22 are connected and furthermore shows the toothed structure of the coupling head 21. Twenty-four teeth 24 are distributed equidistantly along the circumference of the coupling head 21, corresponding to the number of possible rotational positions per revolution of the dose preparation rod 20 relative to the housing 2. The twenty-four teeth 24 allow the dose preparation rod 20 to become rotationally locked relative to the housing 2 by interaction with the interior spline 29 during dose delivery, regardless of its actual rotational position relative to the housing 2. The coupling head 21 has an annular abutment face 25 which serves as a means for engagement with the interior wall 58.

FIGS. 6a and 6b are perspective, respectively longitudinal section views further detailing the rotator 30. As seen, the toothed head 32 holds a number of teeth 34 which are provided for interaction with the central toothed rim portion 82 in a simple gear connection.

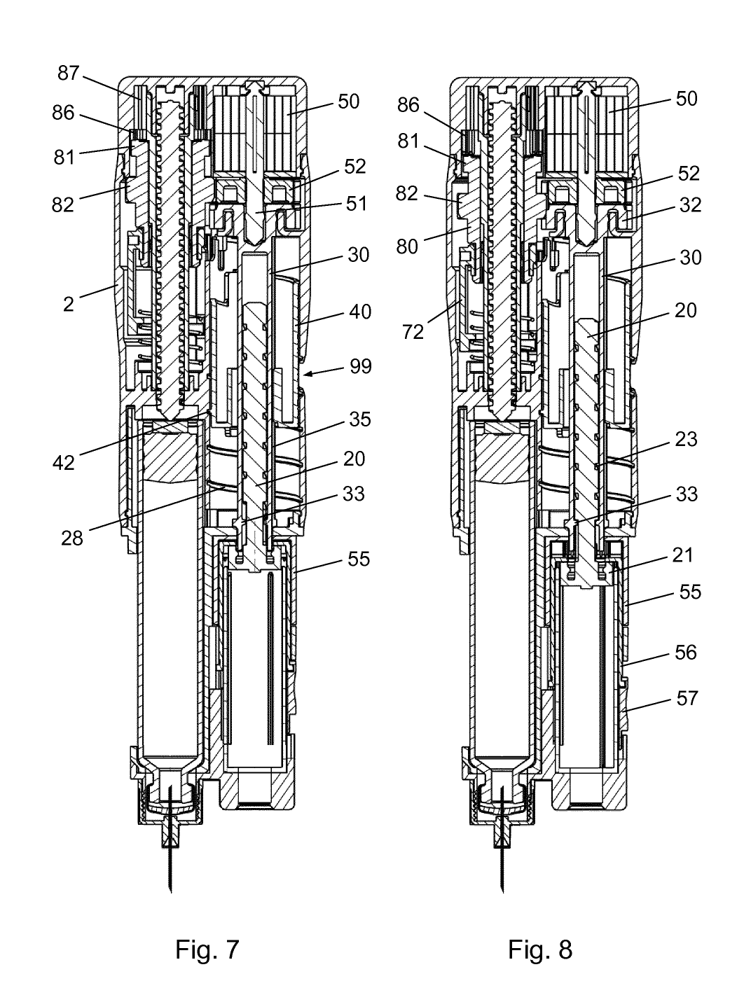

FIGS. 7-12 are longitudinal section views of the injection device 1 in different states during use. In the following a use sequence of the injection device 1 will be explained with reference to these figures.

FIG. 7 shows the injection device 1 in a state just before delivery of the very first dose. The cap 4 has been dismounted from the housing 2 and a dose has been set by rotation of the dose dial 55 about the longitudinal axis defined by the dose preparation rod 20. When the dose dial 55 is rotated the rotational engagement between the teeth 24 and the inner toothed portion of the dose dial 55 causes a joint rotation of the dose preparation rod 20 which due to the interior protrusion 33 residing in the longitudinal groove 22 causes a joint rotation of the rotator 30. Because of the engagement between the axial splines 35 and the longitudinal interior projections 44 the scale drum 40 is also forced to rotate, whereby the scale drum 40 is displaced helically downwards in the housing 2 along the interior thread 28. As the scale drum 40 performs this helical movement relative to the housing 2 the dose indicia 41 sequentially pass by the window 99 to indicate which dose is set. If the user by accident dials too large a dose the direction of rotation of the dose dial 55 is simply reversed, whereby the direction of rotation of the dose preparation rod 20, the rotator 30, and the scale drum 40 is similarly reversed and the scale drum 40 moves helically upwards in the housing 2. The total angular displacement of the dose dial 55 relative to the housing 2 thus correlates with the helical displacement of the scale drum 40 relative to the window 99 and thereby with the actual dose set. The position of the scale drum 40 in FIG. 7 indicates that a dose corresponding to approximately one third of the maximum settable dose has been set.

In FIG. 8 the injection button 57 is slid forward to its distal activated position and the figure shows the injection device 1 in a dose begin state just before the spring 50 releases energy. The distal movement of the injection button 57 causes the carriage 56 to pull the dose preparation rod 20 distally due to the engagement between the interior wall 58 and the abutment face 25. The axial movement of the dose preparation rod 20 causes the teeth 24 to disengage from the dose dial 55 and engage with the interior spline 29 instead, thereby locking the coupling head 21 rotationally to the housing 2. Furthermore, the relative axial motion between the dose preparation rod 20 and the rotator 30 causes the interior protrusion 33 to become positioned at the junction between the longitudinal groove 22 and the helical groove 23. The distal movement of the injection button 57 simultaneously causes the transmission bar 70 to pull the gear 80 distally, against the biasing force from the return spring 65, via the coupling rod 72 and the catch portion 73, whereby the central toothed rim portion 82 firstly moves into additional engagement with the toothed head 32, and the proximal toothed rim portion 81 subsequently disengages from the circumferential toothing 86 and releases the pre-tensioned spring 50.

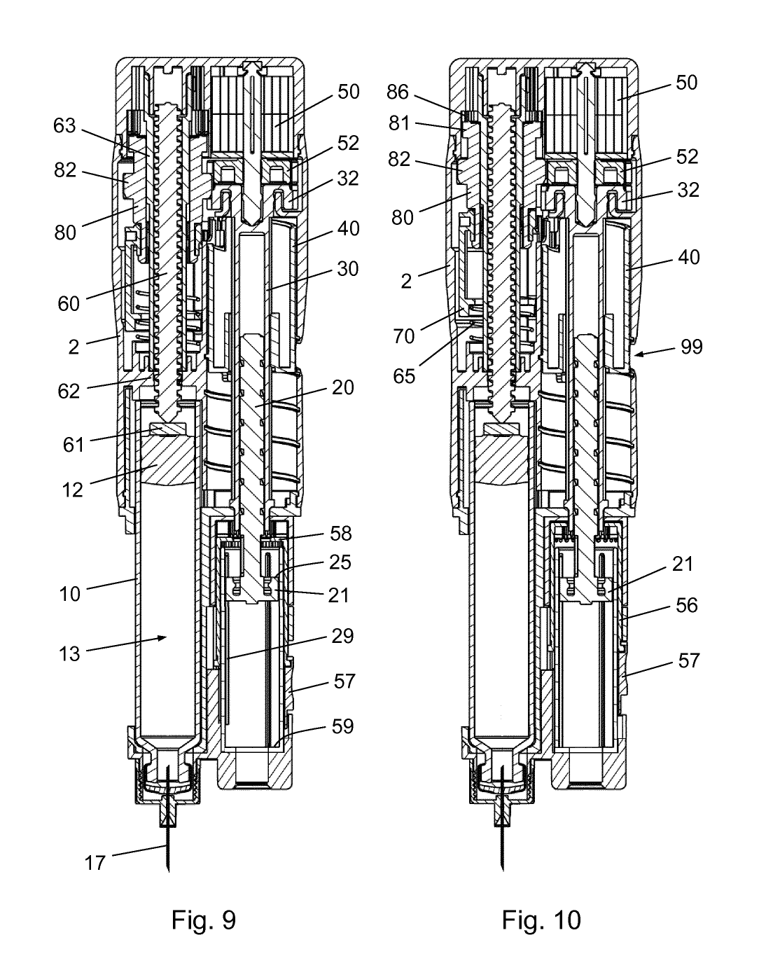

FIG. 9 illustrates that as a result thereof the spring 50 unwinds and the inner spring end portion rotates the spring shaft 51. This rotation is transferred to the toothed head 32 via the central toothed rim portion 82 and as the rotator 30 spins the scale drum 40 is driven upwards along the interior thread 28 towards its zero dose indicating position, while the interior protrusion 33 travels upwards in the helical groove 23 and the dose preparation rod 20 consequently is urged downwards due to its rotational locking engagement with the housing 2.

When a proximal portion of the scale drum 40 rotates into abutment with a proximal stud (not visible) in the housing 2, the scale drum 40 has returned to the zero dose indicating position and therefore cannot rotate further in that direction relative to the housing 2. As a consequence, the rotator 30 stops rotating and the dose preparation rod 20 stops the downwards displacement relative to the housing 2 in an axial position which constitutes an end-of-dose position corresponding to the particular dose delivered. The spring 50 is at this point prevented from releasing further energy, and the injection device 1 is in a “DOSE DELIVERED” state. In principle, the above described respective movements of the scale drum 40 and the dose preparation rod 20 correspond to the respective movements of the dose defining element 40′ and the dose preparation element 20′ sketched in FIG. 1d.

As long as the spring 50 releases energy the gear 80 rotates due to the interaction between the spring shaft 51 and the central toothed rim portion 82, and because the gear 80 has been moved distally by the transmission bar 70, and the interior toothing (not visible) in the gear 80 thereby has moved axially into engagement with the toothed exterior portion (not visible) on the piston rod guide 63, the rotation of the gear 80 is transferred to the piston rod guide 63 and therefrom to the piston rod 60, which due to the engagement with the nut 62 is advanced helically in the distal direction. The piston 12 is thereby pushed into the cartridge 10 to reduce the volume of the drug containing chamber 13 and expel an amount of drug through the injection needle 17. Once the scale drum 40 reaches the zero dose indicating position and the spring 50 is prevented from further unwinding the gear 80 stops rotating and the dose expelling consequently stops. In this position of the scale drum 40 the dose indicia 41 indicate through the window 99 that no dose is prepared for delivery.

By release of the injection button 57, as shown in FIG. 10, the transmission bar 70 and, consequently, the gear 80 are moved proximally by the return spring 65. This causes the central toothed rim portion 82 to disengage from the toothed head 32 and the proximal toothed rim portion 81 to re-engage with the circumferential toothing 86, thereby securing the spring 50. The proximal movement of the transmission bar 70 also causes the injection button 57 to return to its proximal passive position, pushing the carriage 56 along whereby the interior wall 58 is forced into abutment with the dose dial 55.

Notably, however, the dose preparation rod 20 is not moved by this action. The dose preparation rod 20 remains in the end of dose position and is thus still rotationally locked with respect to the housing 2. This means that in the dose delivered state of the injection device 1, where a new dose has not yet been automatically prepared for delivery, a rotation of the dose dial 55 has no effect on the position of the scale drum 40, as the two are decoupled.

It is further noted that in case the user for some reason wishes to pause a dose delivery, at any time during the expelling of drug from the chamber 13, she/he simply releases the injection button 57, whereby the return spring 65 will force the transmission bar 70 and the gear 80 proximally in the housing 2 and cause the proximal toothed rim portion 81 to engage with the circumferential toothing 86 and stop the spring 50 from further unwinding, similar to what is described above. Importantly, as the coupling head 21 becomes disengaged from the dose dial 55 before the spring 50 is released, when the injection button 57 is activated, and does not return when the injection button 57 is released, it is not possible to operate the dose dial 55 to change the dose to be delivered when an injection is paused. Thereby, the user is prevented from adjusting the final set dose in the course of a delivery procedure and thus potentially becoming uncertain of the actual dose set.

FIG. 11 shows the injection device 1 in a “DOSE PREPARED” state after remounting of the cap 4. The remounting motion of the cap 4 relative to the housing 2 causes the actuation rod 9 to exert a proximally directed push force on the coupling head 21 which then causes the dose preparation rod 20 to undergo a reverse, axial displacement back to the dose prepared position along the interior spline 29. This displacement back to the dose prepared position, where the abutment face 25 abuts the interior wall 58, forces the interior protrusion 33 to travel downwards in the helical groove 23 and further into the longitudinal groove 22 back to the position it originally held in the longitudinal groove 22 before the injection button 57 was slid to the activated position. While the interior protrusion 33 travels back down in the helical groove 23 the rotator 30 rotates and slaves the scale drum 40. When the interior protrusion 33 reaches the junction between the helical groove 23 and the longitudinal groove 22 the rotation of the rotator 30 stops, and at that point the scale drum 40 has undergone a helical displacement along the interior thread 28 which is identical in size but opposite in direction to the one it underwent during its movement to the zero dose indicating position, and it has thus been brought back to the same position relative to the housing 2 that it had before the dose delivery was commenced, i.e. the number that can be read through the window 99 equals the dose that was just delivered.

The last part of the relative motion between the dose preparation rod 20 and the rotator 30 during the mounting of the cap 4 onto the injection device 1 is purely axial as the interior protrusion 33 is then positioned in the longitudinal groove 22. In other words, during the last part of the returning displacement of the dose preparation rod 20, where the coupling head 21 disengages from the interior spline 29 and re-engages with the inner toothed portion of the dose dial 55, the scale drum 40 remains stationary relative to the housing 2.

The mounting of the cap 4 onto the cap receiving portion of the housing 2 thus causes a) the dose preparation rod 20 to undergo a displacement relative to the housing 2 which is exactly opposite to the displacement it underwent in the course of the dose delivery procedure, and b) the scale drum 40 to undergo a displacement relative to the housing 2 which is exactly opposite to the displacement it underwent in the course of the dose delivery procedure.

When the cap 4 is mounted on the cap receiving portion after the very first dose delivery the injection device 1 is not only in a “DOSE PREPARED” state but also in a secured or inactive state, because even though a dose has actually been prepared for delivery the side wall 5 covers the injection button 57 which is thus kept inaccessible for operation. Similarly, when the cap 4 is on the dose dial 55 is inaccessible for operation, and the prepared dose can therefore not be adjusted. Consequently, the injection device 1 may be carried about safely in e.g. a bag or pocket without the user risking an inadvertent adjustment of the prepared dose or an administration of the prepared dose to the cap 4.

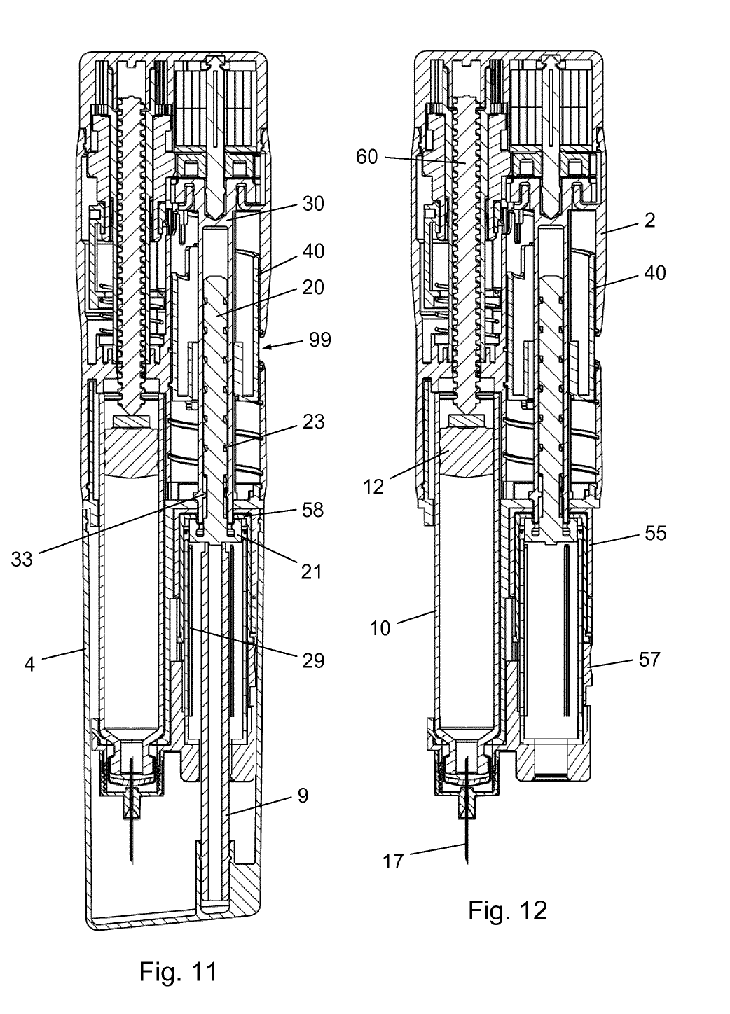

When the dose preparation rod 20 is in the dose prepared position it is snap fitted to the rotator 30 and is thereby axially releasably fixed with respect to the housing 2. It will therefore stay in that position when the user removes the cap 4 before taking the next injection, as illustrated in FIG. 12. After removing the cap 4 the user has two options; in case the dose required in connection with the upcoming injection is the same as the one previously delivered no dose setting actions are needed and the injection needle 17 can simply be inserted into the skin and the injection button 57 slid to the activated position. In case the user needs to adjust the dose, up or down, the dose dial 55 is operable to carry out the adjustment, preferably before the injection needle 17 is inserted into the skin.

As set out in the above, when taking the injection device 1 into use for the first time the user may initially set a dose to be delivered and then only operate the dose dial 55 again in case the initial dose needs to be changed for a subsequent injection. Alternatively, the injection device 1 may be pre-set by a health care professional, or by the manufacturer, in which case the user is free from setting a dose initially, or entirely.

Further, as described in the above, following the very first dose delivery a dose is prepared for delivery automatically in response to the mounting of the cap 4 on the cap receiving portion. It is, however, clear that other means of returning the dose preparation rod 20 to the dose prepared position than the cap 4 may be employed. For example, the actuation rod 9 may be provided as a separate item and may be used by the user to push the coupling head 21 back into abutment with the interior wall 58 independently of the cap 4.

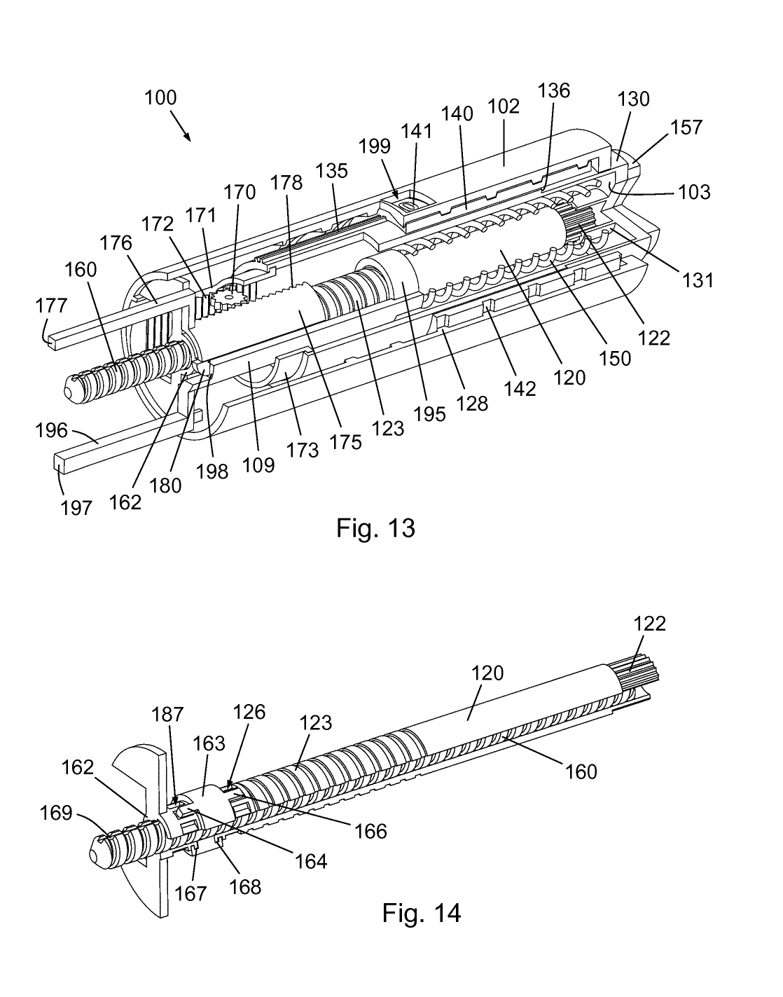

FIG. 13 is a perspective view of a portion of an injection device 100 according to another exemplary embodiment of the invention, specifically of a proximal portion of the injection device 100, carrying a dose engine. The injection device 100 is in a pre-use state and portions of some elements thereof have been removed from the figure to provide a detailed overview of the construction.

The injection device 100 is of the so-called pen injector type and has a tubular housing 102 extending along a longitudinal general axis and accommodating a number of functional components. The housing 102 is coupled with a drug containing cartridge (not shown) in a manner conventionally known in the art, i.a. meaning that the cartridge during use of the injection device 100 is at least axially fixed with respect to the housing 102. Central to the function of the injection device 100 is an axially extending piston rod 160 which is in threaded engagement with a nut 162 that is both axially and rotationally fixed in the housing 102. The distal end portion of the piston rod 160 is coupled to a piston (not shown) in the cartridge such that any advancing axial motion of the piston rod 160 is transferred to the piston, essentially for pressurisation of the cartridge, as is also conventionally known in the art.

It is noted that all rotational movements described in relation to this embodiment of the invention and referred to as clockwise or counter-clockwise are described as seen from the distal end of the piston rod 160 (i.e. from left to right in FIG. 13).

The housing 102 is provided with an interior thread 128 which cooperates with an exterior helical track segment 142 on a scale drum 140, allowing the scale drum 140 to undergo a well-defined helical motion in the housing 102. The scale drum 140 carries a plurality of dose indicia 141 for indicating to a user the particular size of a set dose. The dose indicia 141 are successively viewable through a window 199 in the housing 102 when the scale drum 140 travels along the interior thread 128 e.g. from a proximal “zero dose” position to a distal “maximum dose set” position. The proximal “zero dose” position is defined by a proximal stop surface (not visible) providing a rotational stop for proximal motion of the scale drum 140 at the proximal end of the interior thread 128, whereas the “maximum dose set” position is defined by a distal stop surface (not visible) providing a rotational stop for distal motion of the scale drum 140 at the distal end of the interior thread 128.

The scale drum 140 is rotationally locked to a rotator 130 via a longitudinal interior projection 144 (see FIG. 16) and an axially extending spline 135 on the exterior surface of the rotator 130. While rotationally interlocking the scale drum 140 and the rotator 130 this splined connection allows relative axial motion between the two. The rotator 130 is at its distal end portion axially locked to a coupling piece 173 which comprises an axially aligned leg 171 with a radially inwardly facing toothed surface 172. At the proximal end portion of the rotator 130 a push button 157 is arranged, which is axially locked to but rotationally decoupled from the rotator 130, and the two together serve as an injection button. Further, a sleeve 131 extends axially from an inner end face 103 of the rotator 130. The sleeve 131 has a toothed inner surface and is configured to be brought into and out of rotational interlocking engagement with a toothed end portion 122 of a dose preparation tube 120 which extends axially within the housing 102.

The dose preparation tube 120 has a threaded end portion 123 opposite the toothed end portion 122. The threaded end portion 123 interfaces with a drive nut 195 in a non-self-locking thread engagement. The drive nut 195 forms part of an actuation rod 109, the function of which will be described in detail below. The actuation rod 109, which is axially displaceable but rotationally fixed with respect to the housing 102, has a longitudinal extension 196 which ends in an abutment face 197. The longitudinal extension 196 is transversally offset from a main portion of the actuation rod 109 and is adapted to slide along a cartridge holder (not shown in FIG. 13) both during dose delivery and dose preparation. The cartridge holder is attached to a distal portion of the housing 102 and serves to hold and protect the cartridge in a manner conventionally known in the art.

A pre-tensioned compression spring 150 is arranged to act between the inner end face 103 and the actuation rod 109, constantly biasing the rotator 130 and the push button 157 proximalty, out of the housing 102, and the actuation rod 109 distally. In the shown pre-use state of the injection device 100 distal motion of the actuation rod 109 is prevented by a lock member 180 abutting a transversal surface 198 of the actuation rod 109. The lock member 180 is pivotally arranged on the nut 162 but is in FIG. 13 prevented from pivoting by an edge portion of a button coupling rod 175 which is axially displaceable but rotationally fixed with respect to the housing 102. The button coupling rod 175 has a toothed straight edge 178, which is in engagement with a transmission wheel 170, and a longitudinal extension 176, which is transversally offset from the toothed straight edge 178 and which ends in an abutment face 177. The transmission wheel 170 is further in engagement with the toothed surface 172, such that the coupling piece 173, the button coupling rod 175, and the transmission wheel 170 together provide a double rack and pinion drive.

In the present situation, given that the actuation rod 109 is prevented from undergoing distal motion in the housing 102 due to the lock member 180, the bias of the spring 150 on the rotator 130 causes the rotator 130 to exert a pulling force on the coupling piece 173 which then via the double rack and pinion arrangement is converted to a distal movement of the button coupling rod 175, unless a counter-acting force is applied to the abutment face 177. Although not shown in FIG. 13, in the depicted pre-use state of the injection device 100 a removable protective cap is securely mounted onto a cap receiving portion at the distal end portion of the housing 102 such that a portion of the cap abuts the abutment face 177 and resists the bias conveyed to the longitudinal extension 176, thereby maintaining the button coupling rod 175 in position. The injection device 100 is thus in fact stably locked in a tensioned state. As will be explained in more detail below once the retaining force on the abutment face 177 is removed the relaxation of the spring 150 will cause the rotator 130 and the push button 157 to translate proximally. A stop surface 136 on the rotator 130 limits the proximal motion of the rotator 130 and the push button 157 relative to the housing 102.

FIG. 14 is a detailed view of the piston rod advancement mechanism as employed in the injection device 100. A rotatable piston rod guide 163 couples the nut 162 and the dose preparation tube 120 via an inner groove 167 for axial interlocking connection with the nut 162 and an inner groove 168 for axial interlocking connection with the threaded end portion 123. The piston rod guide 163 has a distal pawl 164, which in combination with a plurality of circumferentially spaced apart indentations 187 on the nut 162 provide a distal ratchet mechanism, and a proximal pawl 166 which in combination with a plurality of circumferentially spaced apart indentations 126 on the dose preparation tube 120 provide a proximal ratchet mechanism. The distal ratchet mechanism allows clockwise rotation of the piston rod guide 163 relative to the nut 162 but prevents counter-clockwise rotation of the piston rod guide 163. The proximal ratchet mechanism allows relative rotation between the dose preparation tube 120 and the piston rod guide 163 when the dose preparation tube 120 is rotated counter-clockwise, but prevents relative rotation between the dose preparation tube 120 and the piston rod guide 163 when the dose preparation tube 120 is rotated clockwise. The double ratchet comprised of the distal ratchet mechanism and the proximal ratchet mechanism thus allows the dose preparation tube 120 to drag the piston rod guide 163 along in the clockwise direction and to rotate freely in the counter-clockwise direction while the piston rod guide 163 remains stationary.

The piston rod guide 163 further has a radially inwardly directed protrusion (not visible) for engagement with an axial groove 169 on the piston rod 160. The piston rod 160 and the piston rod guide 163 are thus rotationally interlocked but capable of relative axial motion.

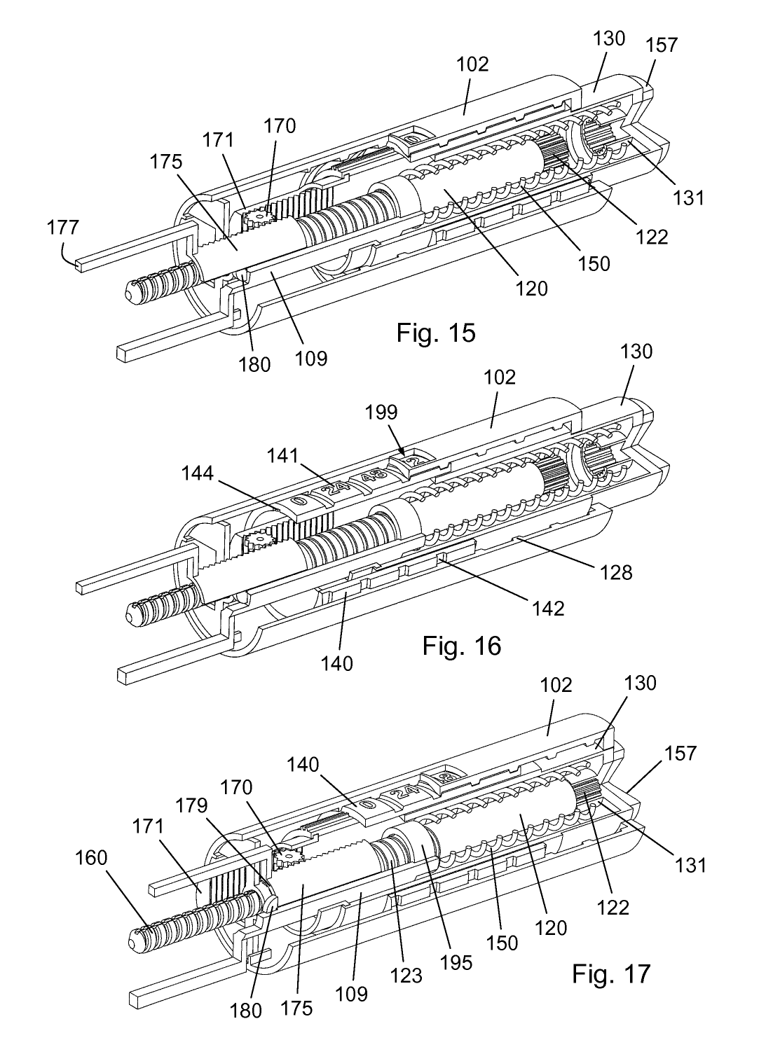

The functionality of the dose setting and delivery mechanisms will now be described with reference to FIGS. 15-19. When taking the injection device 100 into use the protective cap is firstly removed. This removes the retaining force on the abutment face 177 and allows the spring 150 to expand. The spring 150 thus urges the rotator 130 with the push button 157 proximally until the stop surface 136 abuts the interior end wall of the housing 102, and the double rack and pinion drive accordingly forces the button coupling rod 175 a distance distally. The end result of this is illustrated by FIG. 15.

The proximal motion of the rotator 130 also causes the sleeve 131 to disengage from the toothed end portion 122. The rotator 130 is thus now capable of being rotated without affecting the dose preparation tube 120. A dose is set by rotation of the rotator 130 relative to the housing 102. Due to the spline connection between the rotator 130 and the scale drum 140 and the threaded interface between the scale drum 140 and the housing 102 when the rotator 130 is dialed counter-clockwise the scale drum 140 displaces helically downwards in the housing 102 in response, and when the rotator 130 is dialed clockwise the scale drum 140 displaces helically upwards in the housing 102. In FIG. 16 the rotator 130 has been dialed to set a dose of “72” units.

Dose delivery is executed by depression of the push button 157, as illustrated in FIG. 17. The push button 157 may actually be depressed a certain distance without causing more than a reversed motion of the double rack and pinion drive and a compression of the spring 150. A discontinuation of the depression force in this instance will simply cause the spring 150 to return the push button 157 to its proximal most position. However, once the button coupling rod 175, during its proximal displacement, reaches a specific axial position in the housing 102 an end surface 179 passes the fulcrum of the lock member 180 and the lock member 180 will be free to pivot, whereby the pre-tensioned spring 150 will be released and as a result force the actuation rod 109 distally. As the lock member 180 pivots to allow passage of the actuation rod 109 the button coupling rod 175 becomes prevented from distal motion in the housing 102 due to the lock member 180 being prevented from returning to the original position by the actuation rod 109. At this point if the user releases the pressure on the push button 157 the rotator 130 will consequently be prevented from proximal motion and will thus stay inside the housing 102.

The depression of the push button 157 also leads to a rotational re-engagement of the sleeve 131 and the toothed end portion 122. This happens before the flipping over of the lock member 180, such that when the spring 150 is released and the actuation rod 109 is suddenly propelled distally the rotator 130 and the dose preparation tube 120 are rotationally interlocked. Due to the threaded engagement between the drive nut 195 and the threaded end portion 123 the distal movement of the actuation rod 109 causes the dose preparation tube 120 to spin clockwise.

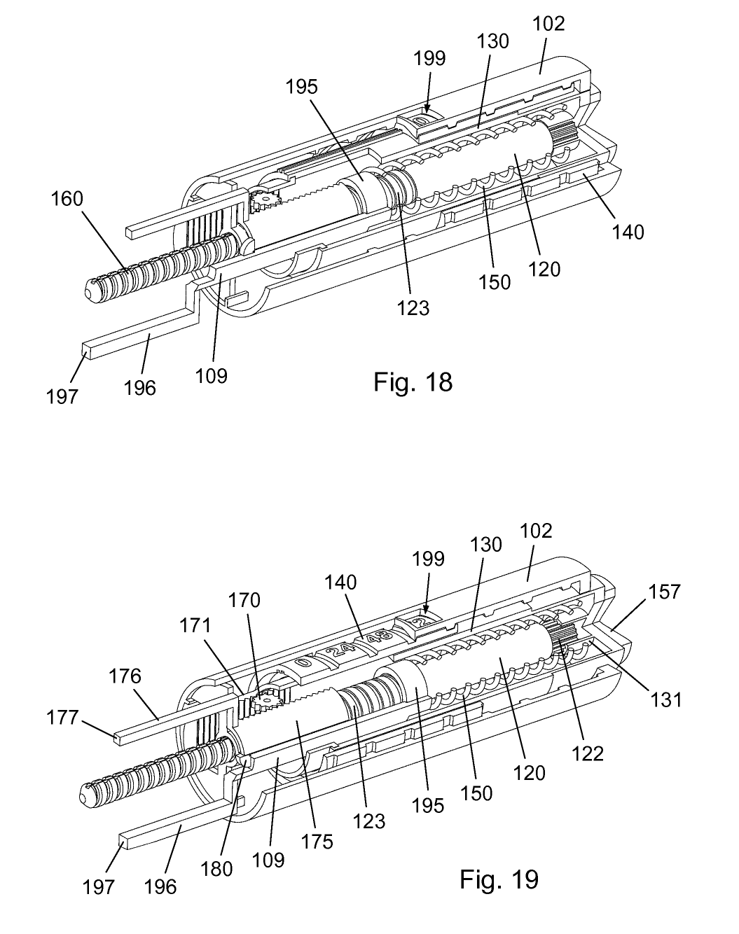

The clockwise rotation of the dose preparation tube 120 causes a clockwise rotation of the piston rod guide 163, due to the above described double ratchet mechanism, and thereby also of the piston rod 160. The threaded engagement between the piston rod 160 and the nut 162 thus results in a helical advancement of the piston rod 160, whereby the piston (not shown) is advanced axially in the cartridge (not shown) to expel a volume of drug through an attached injection needle (not shown). The volume expelled is determined by the position of the scale drum 140 in the housing 102 at the time of release of the spring 150 because the clockwise rotation of the dose preparation tube 120 also causes a clockwise rotation of the rotator 130 and thereby of the scale drum 140, and the rotation of the three continues until the scale drum 140 meets the proximal stop surface which defines the “zero dose” position. This end-of-dose state of the injection device 100 is illustrated in FIG. 18.

It is noted that as the injection progresses the actuation rod 109 is moved further distally and the axial end position of the abutment face 197 corresponding to the “zero dose” position of the scale drum 140 is uniquely correlated with the distance traveled by the scale drum 140 from its position at release of the spring 150 to the proximal stop surface.

In the end-of-dose state of the injection device 100 the push button 157 is prevented from proximal motion and therefore has to stay depressed in the housing 102. Hence, it is not possible to set a dose at this point. It is common practice when handling injection devices to re-mount the protective cap following an injection. In the course of re-mounting the protective cap onto the cap receiving portion of the injection device 100 a portion of the cap, such as e.g. a segment of the cap rim or a protrusion, abuts the abutment face 197 and pushes the actuation rod 109 proximally with respect to the housing 102.

The resulting proximal movement of the drive nut 195 causes the dose preparation tube 120 to spin counter-clockwise, relative to the housing 102 but also relative to the piston rod guide 163 due to the double ratchet mechanism, so the piston rod 160 is left unaffected. The counter-clockwise rotation of the dose preparation tube 120 causes a corresponding counter-clockwise rotation of the rotator 130 which leads to a downward helical displacement of the scale drum 140.

The proximal movement of the drive nut 195 also causes a compression of the spring 150 which is progressive until the actuation rod 109 reaches the axial position where the transversal surface 198 passes the fulcrum of the lock member 180. At this position of the actuation rod 109 the lock member 180 is free to pivot and thus no longer functions as a block for distal motion of the button coupling rod 175. So, as the spring 150 seeks to relax and constantly biases the inner end face 103 in the proximal direction, the rotator 130 is urged proximally, pulling the coupling piece 173, and the double rack and pinion drive consequently urges the button coupling rod 175 distally, causing the lock member 180 to flip over and abut the transversal surface 198. The spring 150 will displace the rotator 130 proximally a small distance until the abutment face 177 abuts the protective cap and further distal motion of the button coupling rod 175 thereby is prevented. This corresponds to the state of the injection device 100 shown in FIG. 19. In this state the lock member 180 stably prevents distal motion of the actuation rod 109, as it is prevented from pivoting by the button coupling rod 175. As long as the protective cap remains mounted on the cap receiving portion a depression of the push button 157 only leads to an additional compression of the spring 150 which has no effect on the secured injection mechanism. At termination of the push force the spring 150 will return to the slightly less compressed state shown in FIG. 19.

Notably, when the protective cap is re-mounted on the cap receiving portion the actuation rod 109 is returned to the exact same axial position within the housing 102 that it initially assumed before the dose ejection was commenced. Due to the threaded interface between the drive nut 195 and the threaded end portion 123 this means that the dose preparation tube 120 is consequently returned rotationally to the exact same angular position relative to the housing 102 that it initially assumed before the dose ejection was commenced. The dose preparation tube 120 has thus during re-mounting of the protective cap undergone the exact opposite rotation to the one it underwent during the dose delivery, and since the dose preparation tube 120 and the rotator 130 are rotationally interlocked so has the rotator 130. Consequently, due to the splined connection between the rotator 130 and the scale drum 140 and the threaded connection between the scale drum 140 and the housing 102, the scale drum 140 has been returned to the position it assumed immediately before the push button 157 was depressed and the spring 150 was released. In other words, by the re-mounting of the protective cap onto the cap receiving portion a setting of the last ejected dose has automatically been performed.

In fact, every time the protective cap is mounted onto the cap receiving portion the dose preparation tube 120 will be returned, in the above described manner, to the initial angular position, which can be defined as a dose prepared position within the housing 102, thereby bringing the injection device 100 in a “DOSE PREPARED” state.

When the user dismounts the protective cap before the next injection the rotator 130 and the push button 157 will re-protrude from the housing 102 and the sleeve 131 will disengage from the toothed end portion 122, as described above in connection with FIG. 15. The user can now either choose to simply position the injection device 100 at the desired skin site and press the push button 157 to deliver the same dose as was last delivered, or adjust the dose size by dialing the rotator 130 in the appropriate direction before performing the injection procedure.

In case the user chooses to adjust the dose, and thereby set a new dose, the scale drum 140 will change position within the housing 102 and assume a new position corresponding to the new dose viewed through the window 199. Because the rotator 130 is decoupled from the toothed end portion 122 the repositioning of the scale drum 140 will not affect the dose preparation tube 120. Only when the push button 157 is subsequently depressed and the sleeve 131 reengages with the toothed end portion 122 the scale drum 140 and the dose preparation tube 120 become coupled to undergo correlated movements relative to the housing 102, provoked by the spring 150, as previously described. During these correlated movements the scale drum 140 will again reach the “zero dose” position and abruptly stop further expansion of the spring 150 and distal motion of the actuation rod 109. When this happens the axial end-of-dose position of the abutment face 197 relative to the housing 102 will be different from its previous end-of-dose position and, consequently, the dose preparation tube 120 will have undergone a different angular displacement than the one it underwent during the previous dose delivery. Nevertheless, when the cap is re-mounted on the cap receiving portion the actuation rod 109 will once again be returned to the same axial position as before, since that axial position is defined by the position of the cap portion abutting the abutment face 197 relative to the housing 102 when the cap is securely mounted. Due to the engagement between the drive nut 195 and the threaded end portion 123 the reversed motion of the actuation rod 109 will lead to a reversed motion of the dose preparation tube 120, which will again lead to a reversed motion of the scale drum 140. Thereby, the dose preparation tube 120 is returned to the exact same angular position relative to the housing 102 that it assumed before the dose ejection (the dose prepared position), and the scale drum 140 is returned to the position in which the new dose is viewed through the window 199.

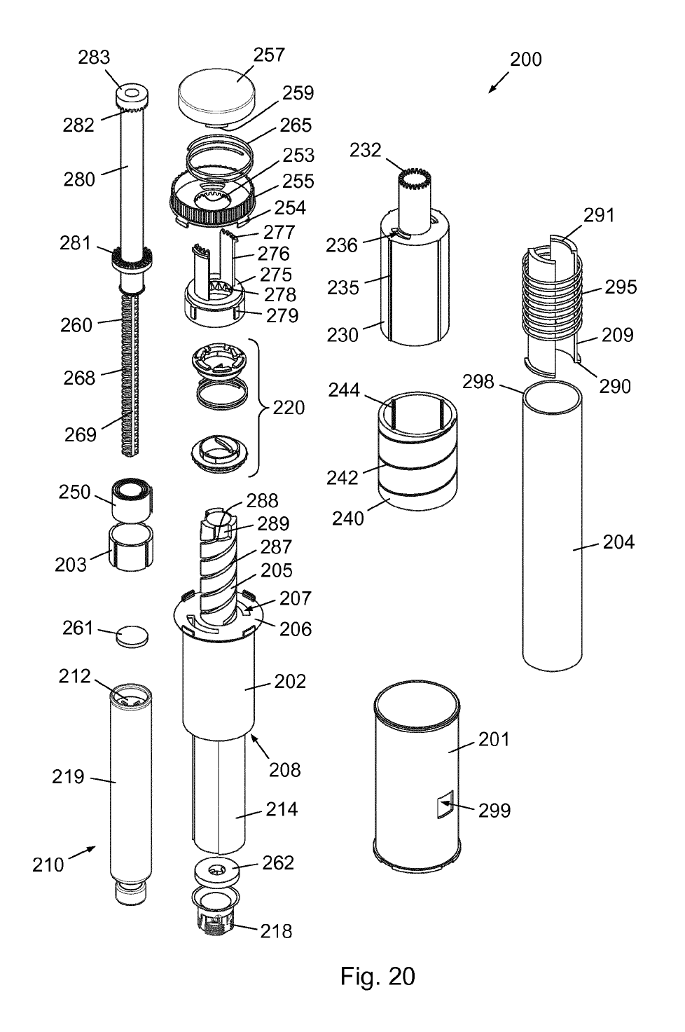

FIG. 20 is an exploded view of a drug delivery device according to yet another exemplary embodiment of the present invention. The drug delivery device is in the form of an automatic injection device 200 which is adapted to deliver set doses of drug from a cartridge 210. The injection device 200 comprises a proximal housing part 201 and a distal housing part 202 which are connected to form a unitary exterior cabinet of a generally tubular configuration. The distal housing part 202 has a distally extending cartridge holder 214 configured to hold and protect the cartridge 210, and to which a needle mount 218 for receiving a needle assembly (not shown) is attached, a transversal partition 206, and a proximally extending hollow spindle 205 serving as a guide structure for a nut assembly 220.

The spindle 205 comprises an exterior helical track 287 which leads into a longitudinal track 289 at a transition point 288. The helical track 287 is configured to be non-self-locking which means that its pitch enables a helical displacement of a mating nut structure under the influence of a purely axial external force. The transversal partition 206 has a pair of narrow slots 207, each slot 207 being configured to receive and to allow longitudinal displacement of a respective loading rod 209. Each loading rod 209 has a distal abutment edge 290 for interaction with a rim 298 of a protective cap 204 when the cap 204 is inserted into a cap receiving portion 208 of the distal housing part 202, and a proximal catch portion 291 for interaction with the nut assembly 220 in a manner that will be described in more detail in the following. A reset spring 295 is arranged to act between the distal abutment edge 290 and a distally facing portion of the transversal partition 206.

The nut assembly 220 is connected to a nut connector 275 which is an intermediate coupling element in a dose setting mechanism of the injection device 200. The nut connector 275 has an annular base from which two connecting arms 276 extend proximally. Each connecting arm 276 is provided with a toothing 277 at its free end. The base of the nut connector 275 has a circumferential set of teeth 278 on an interior surface portion and a plurality of protrusions 279 distributed along an exterior surface portion.

The toothing 277 on each arm 276 is adapted for rotational interlocking engagement with a mating toothed rim 253 of a dose dial 255. The dose dial 255 is connected to the proximal housing part 201 via a number of snap locks 254 and is configured to accommodate an injection button 257 in a way as to allow the injection button 257 to move axially up and down under influence of a button spring 265. The injection button 257 has a distally directed protrusion 259 for interaction with the nut connector 275 in a manner which will be further described in the below.

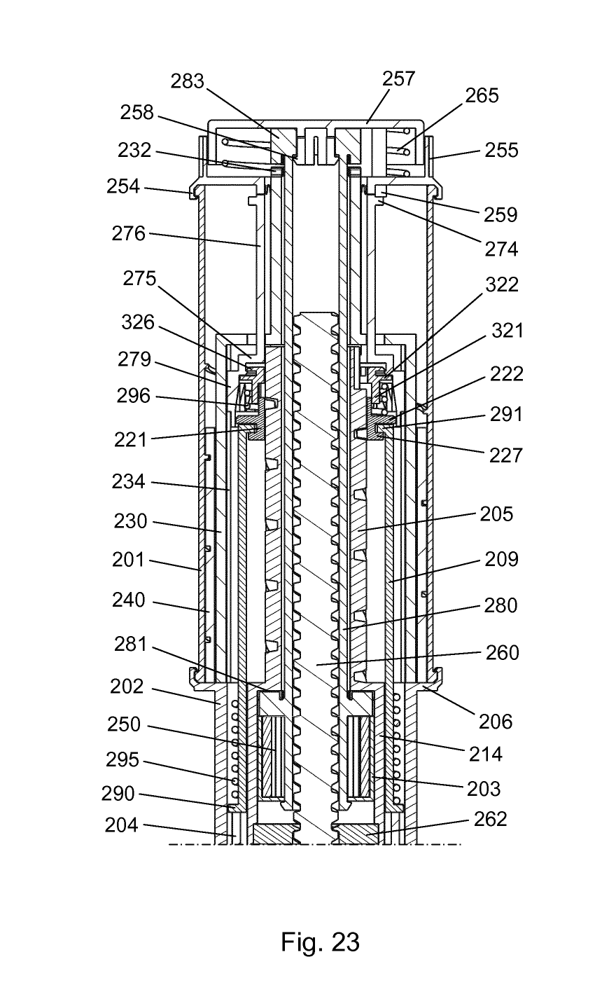

The protrusions 279 are received in respective longitudinal tracks 234 (see FIG. 23) in an interior surface of a scale drum connector 230, providing for an axially free but rotationally interlocked relation between the nut connector 275 and the scale drum connector 230. Openings 236 are provided in a transversal portion of the scale drum connector 230 to allow passage of the arms 276, and a toothed rim 232 is provided at the proximal end. On the exterior surface of the scale drum connector 230 a plurality of splines 235 are distributed which are in engagement with respective splines 244 in a scale drum 240.

The scale drum 240 is a tubular structure having a helically extending groove 242 for threaded engagement with an interior wall portion of the proximal housing part 201 as well as a plurality of dose related numerals (not shown) printed in a helical path on its exterior surface. The scale drum 240 is thus rotationally locked to the scale drum connector 230 and bound to move helically with respect to the proximal housing part 201 in response to a rotation of the scale drum connector 230. In every possible angular position of the scale drum 240 relative to the proximal housing part 201 at least one dose related numeral will be viewable through a window 299, allowing the user to easily identify the size of a set dose.

A driver 280 for effecting the dose delivery is slidingly arranged in the hollow of the spindle 205. The driver 280 has an elongated tubular body with a driver head 283 at a proximal end and a toothed transversal surface 281 arranged at a distal position. The driver head 283 has a distally oriented toothed rim 282 for rotational interlocking engagement with the toothed rim 232 during dose delivery, and the toothed transversal surface 281 is adapted to releasably engage with a mating toothing (not visible) on a distally facing portion of the partition 206, providing for a releasable rotational interlocking connection between the driver 280 and the distal housing portion 202.

The distal most portion of the driver 280 is connected to an inner free end of a spiral spring 250. An outer free end of the spiral spring 250 is connected to an interior surface of a spring housing 203, which spring housing 203 is rotationally locked in the distal housing part 202.

The spiral spring 250 is pre-strained and has sufficient capacity to cause a complete emptying of the cartridge 210.

The driver 280 accommodates a piston rod 260 which has a thread 268 for interaction with a guide nut 262 that is fixedly arranged in the distal housing part 202. The thread 268 is interrupted by a longitudinal track 269 extending along the entire length of the piston rod 260. This provides for a splined connection between a protrusion (not visible) on an inner surface portion of the driver 280 and the longitudinal track 269, which ensures that any angular displacement of the driver 280 is passed on to the piston rod 260.

A distal end of the piston rod 260 abuts a piston washer 261 which again abuts a proximal surface portion of an axially displaceable piston 212 arranged in sealing connection with a cartridge wall 219. A counter-clockwise rotation of the driver 280, initiated for dose delivery, will thus cause a counter-clockwise rotation of the piston rod 260 which due to the threaded engagement with the guide nut 262 will be advanced helically downwards, pressing the piston washer 261 and the piston 212 into the cartridge 210 for expelling of a volume of drug through an injection needle (not shown) of a mounted needle assembly.

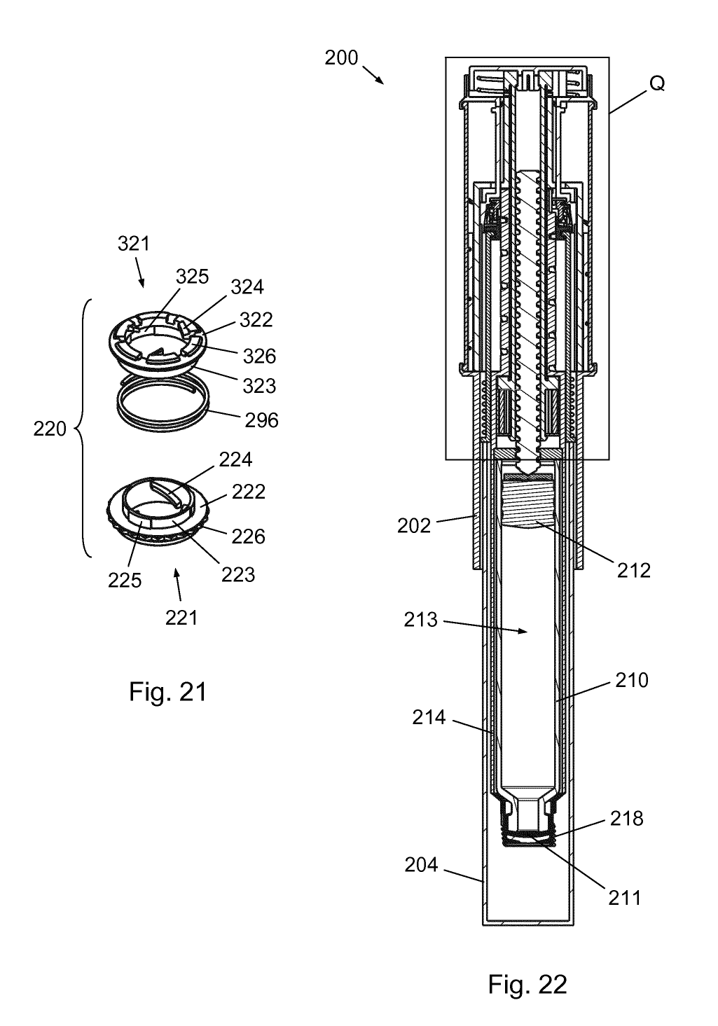

FIG. 21 is an exploded view detailing the nut assembly 220. The nut assembly 220 comprises a primary nut 221, a lock nut 321, and a nut spring 296. The primary nut 221 comprises a transversal spring base 222 which is provided with a round-going set of teeth 226 at its periphery, and a cylindrical portion 223 which has an inwardly projecting helical segment 224 for mating engagement with the helical track 287 and a plurality of circumferentially distributed exterior protuberances 225. The set of teeth 226 is configured to enable releasable engagement with the set of teeth 278, to provide for a rotational interlocking connection between the primary nut 221 and the nut connector 275 in a first relative axial position of the two and a rotational decoupling in a second relative axial position of the two.

The lock nut 321 comprises an annular base 322, from which a number of hook members 326 project, and a cylindrical portion 323 which has a plurality of indentations 325 distributed along an inner surface. Each indentation 325 is configured for sliding reception of one protuberance 225 to ensure a rotational interlocking connection between the primary nut 221 and the lock nut 321. An inwardly directed protrusion 324 on the lock nut 321 is configured for mating engagement with the helical track 287 in one state of the nut assembly 220 and for engagement with the longitudinal track 289 in another state of the nut assembly 220. The hook members 326 are configured to axially retain an interior edge of the nut connector 275 and thereby prevent relative axial movement, while allowing relative rotation, between the nut connector 275 and the lock nut 321. The nut spring 296 is arranged to act between the spring base 222 and the annular base 322, biasing the primary nut 221 and the lock nut 321 axially away from one another.

FIG. 22 is a longitudinal section view of the injection device 200 in a dose setting state before a needle assembly has been mounted on the needle mount 218. The cap 204 is removably mounted on the injection device 200 to cover and protect the cartridge 210. The cartridge 210 holds a drug substance (not visible) in a variable volume chamber 213 which is bounded by the cartridge wall 219, the slidable piston 212, and a penetrable self-sealing septum 211.

FIG. 23 is a close-up view of a proximal portion of the injection device 200 delimited by the area Q in FIG. 22. The figure shows details of the various components and their respective connections with other components in the dose setting state of the injection device 200. In particular, the figure shows the nut assembly 220 in its axially expanded state, being immobilised on the spindle 205, i.e. where the protrusion 324 is in engagement with the longitudinal track 289, and where the primary nut 221 and the lock nut 321 are axially spaced apart by the nut spring 296. This can be termed a top position, or a dose prepared position, of the nut assembly 220. Notably, after removal of the cap 204 the nut assembly 220 will remain in the top position against the biasing axial force from the reset spring 295 acting on the primary nut 221 via the loading rods 209, which are axially fixed between the spring base 222 and a lower rim 227, because the lock nut 321 is prevented from rotating relative to the spindle 205 due to the engagement with the longitudinal track 289.

The injection button 257 is in its non-activated position relative to the proximal housing part 201, which means that the protrusion 259 rests against a rim 274 at the proximal end of one of the arms 276 without applying any significant force thereto, and the toothing 277 is in engagement with the toothed rim 253. A rotation of the dose dial 255 will thus cause a rotation of the nut connector 275 which due to the rotational interaction between the protrusions 279 and the longitudinal tracks 234 will cause a rotation of the scale drum connector 230. The rotation of the scale drum connector 230 is then transferred to the scale drum 240 due to the splined relationship between the two. Hence, when the nut assembly 220 is in the top position any angular displacement of the dose dial 255 will lead to a similar angular displacement of the scale drum 240. Due to the thread connection between the helically extending groove 242 and the proximal housing part 201 an angular displacement of the dose dial 255 will in fact lead to a combined angular and axial displacement of the scale drum 240. This displacement of the scale drum 240 relative to the proximal housing part 201 from a zero dose indicating position correlates directly with the size of the set dose which can be read through the window 299.

The injection button 257 is biased towards the non-activated position by the button spring 265 and the driver 280 is accordingly biased towards a proximal position due to the driver head 283 being axially retained by a retainer member 258. Thus, in the non-activated position of the injection button 257 the toothed transversal surface 281 is held firmly in rotational interlocking connection with the partition 206, and the spring 250 is thereby safely cocked.

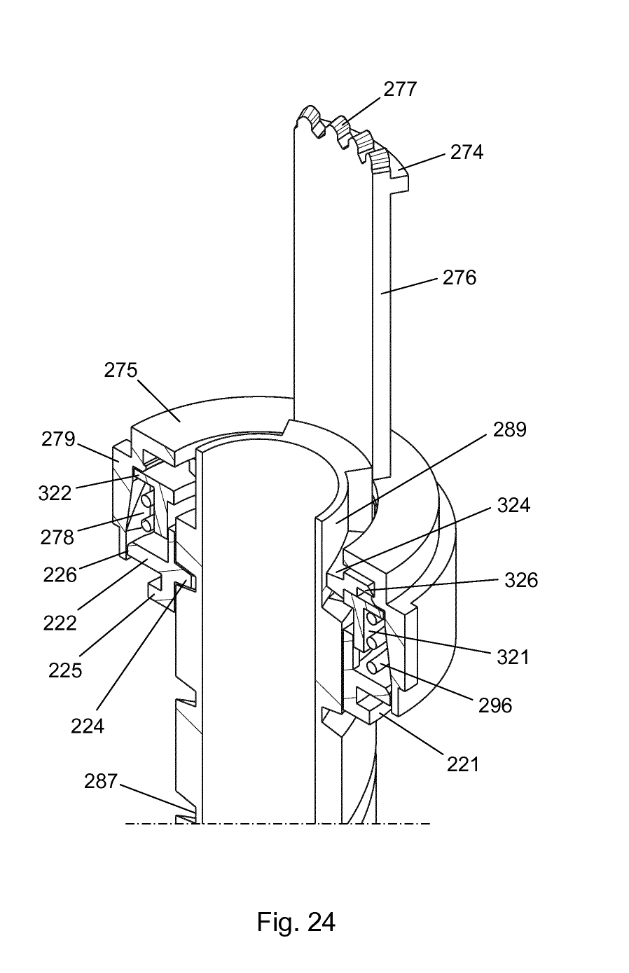

FIG. 24 is a perspective section view of a proximal portion of the spindle 205 showing the nut assembly 220 in the top position. Here, it can be seen more clearly that the helical segment 224 is in engagement with the helical track 287, while the protrusion 324 is in engagement with the longitudinal track 289, effectively preventing any rotation of the lock nut 321 and any movement at all of the primary nut 221 due to the engagement between the respective protuberances 225 and indentations 325 providing the rotational interlocking connection between the primary nut 221 and the lock nut 321. Further, it is seen that the relative axial position of the primary nut 221 and the nut connector 275 is such that the set of teeth 226 on the spring base 222 is disengaged from the set of teeth 278 on the interior surface of the base of the nut connector 275, whereby the nut connector 275 is capable of rotation with respect to the nut assembly 220.

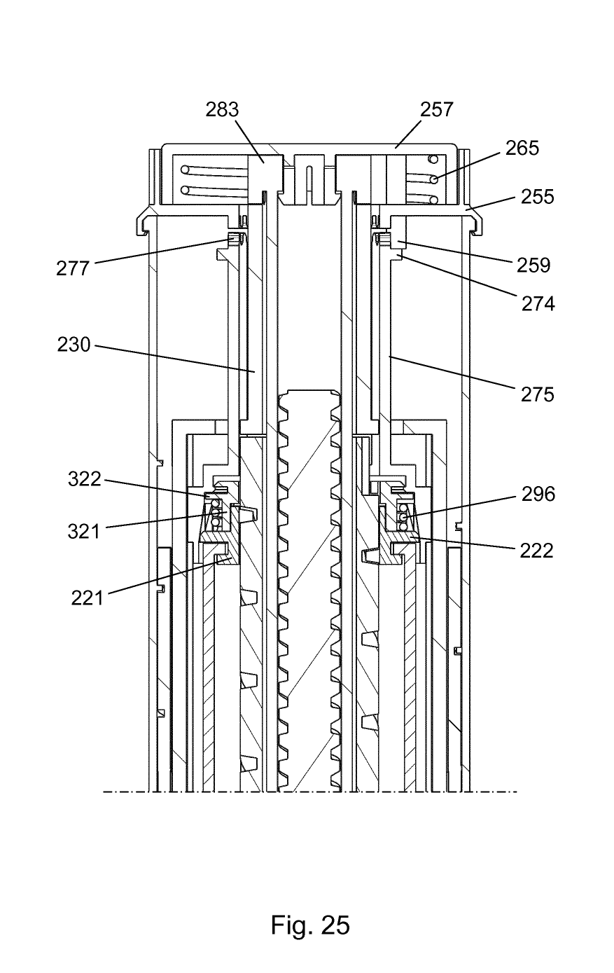

FIG. 25 shows a proximal portion of the injection device 200 and illustrates what happens when the injection button 257 is being depressed. Apart from a compression of the button spring 265 a depression of the injection button 257 entails four major changes to the interrelations between certain components. Firstly, the rim 274 is forced downwards by the protrusion 259 causing the toothing 277 to disengage from the toothed rim 253. The dose setting mechanism is thereby disabled because of the resulting decoupling of the scale drum 240 from the dose dial 205. Secondly, the lock nut 321 is forced downwards by the nut connector 275 exerting a pushing force on the annular base 322. This compresses the nut spring 296 and brings the lock nut 321 to the transition point 288. Thirdly, the driver head 283 is forced downwards, whereby the toothed rim 282 is brought into engagement with the toothed rim 232 on the scale drum connector 230, rotationally coupling the driver 280 and the scale drum 240. Fourthly, the downward movement of the driver 280 brings the toothed transversal surface 281 out of the rotational locking engagement with the partition 206, and the spring 250 is thus released.

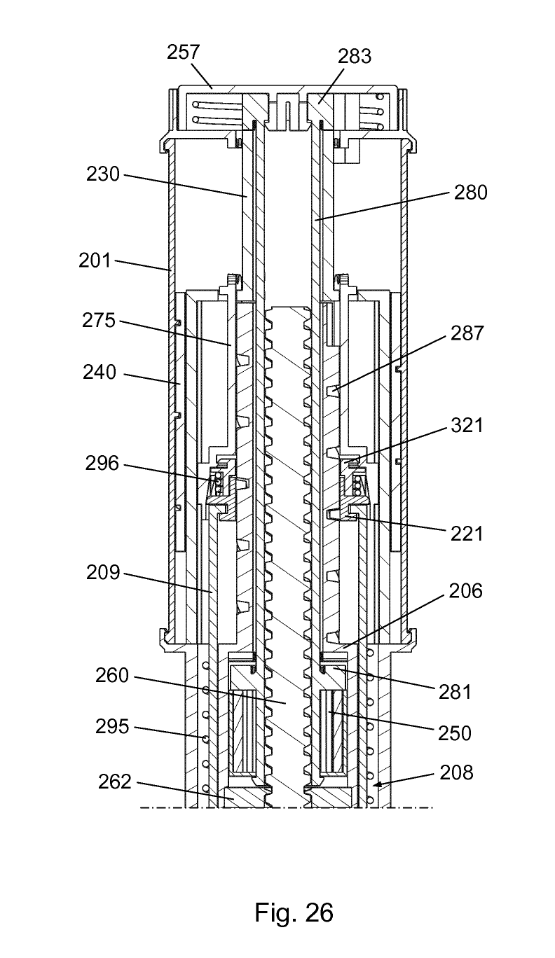

FIG. 26 shows the effect of the release of the spring 250. When the toothed transversal surface 281 is no longer prevented from angular displacement relative to the partition 206 the spring 250 is free to release stored energy for rotation of the driver 280. This causes a corresponding rotation of the piston rod 260 which due to the threaded engagement with the guide nut 262 is advanced helically through the distal housing part 202, forcing the piston 212 along the cartridge wall 219. As the driver head 283 rotates the scale drum connector 230 and the scale drum 240 rotate, which causes a helical upwards movement of the scale drum 240 in the proximal housing part 201.

The spring 250 will release energy and rotate the dose delivery components until the scale drum 240 reaches a physical stop surface in the proximal housing part 201 signifying an end-of-dose state of the injection device 200. The total angular displacement of the scale drum 240 relative to the proximal housing part 201 during the release of energy from the spring 250 correlates with the delivered amount of drug substance from the chamber 213, and in the end-of-dose state the scale drum 240 is in a zero dose indicating position. A subsequent removal of the depressing force from the injection button 257 will cause the button spring 265 to return the injection button 257 to the non-activated position, bringing along the driver head 283 and thereby decoupling the toothed rim 282 from the toothed rim 232 and moving the toothed transversal surface 281 back into rotational interlocking engagement with the partition 206.

Returning to the effect of the release of the spring 250, when the lock nut 321 is positioned at the transition point 288 the set of teeth 278 on the interior surface of the nut connector 275 has moved into engagement with the set of teeth 226 on the circumference of the spring base 222, and the nut connector 275 is thereby rotationally locked to the primary nut 221. The spring induced rotation of the driver 280 which is transferred to the scale drum connector 230 is thus also transferred to the nut connector 275, due to the engagement between the respective protrusions 279 and longitudinal tracks 234, and to the nut assembly 220. Accordingly, the protrusion 324 enters into the helical track 287 and the nut assembly 220 as a unit is displaced helically down the spindle 205 since now both the primary nut 221 and the lock nut 321 are in engagement with the helical track 287. This helical displacement of the nut assembly 220 continues as long as the driver 280 rotates. As the primary nut 221 is thereby moved down towards the partition 206, bringing along the loading rods 209, the reset spring 295 is allowed to expand.

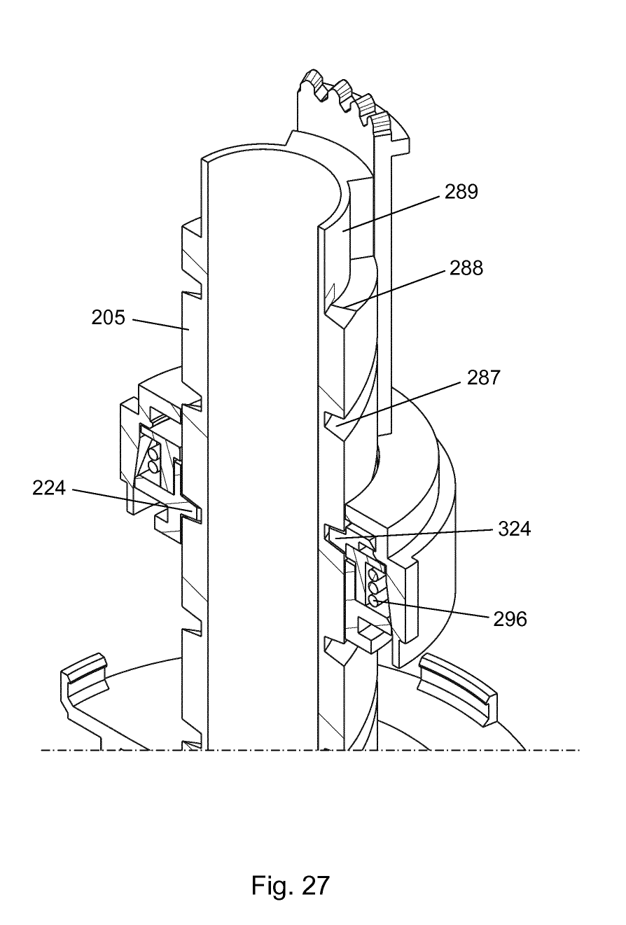

FIG. 27 shows a perspective section view of the nut assembly 220 in the same position as the one depicted by FIG. 26. The figure shows the nut assembly 220 in its axially compressed state, and it is clearly seen that both the helical segment 224 and the protrusion 324 are in engagement with the longitudinal track 287.

Following a completed dose delivery when the user re-mounts the cap 204 onto the injection device 200 the rim 298 of the cap 204 abuts the abutment edge 290 on the respective loading rods 209 and forces the loading rods 209 towards the proximal end of the proximal housing part 201 against the biasing force from the reset spring 295. Thereby, the respective catch portions 291 act on the spring base 222 and push the primary nut 221 in the same direction, while the reset spring 295 is compressed. Due to the helical segment 224 being in engagement with the helical track 287 and the rotational interlocking connection between the respective protuberances 225 and indentations 325 the entire nut assembly 220, in its compressed state, travels helically up the spindle 205 towards the transition point 288.

The resulting angular displacement of the primary nut 221 is transferred to the nut connector 275 and further on to the scale drum connector 230 and the scale drum 240, causing the scale drum 240 to move helically in the proximal housing part 201 away from the zero dose indicating position for automatic setting of a dose which corresponds to the dose that was just delivered. If during the travel of the nut assembly 220 up the spindle 205 the user suddenly chooses to remove the cap 204 the reset spring 295 will expand and drive the nut assembly 220 back down the spindle 205, causing an opposite angular displacement of the primary nut 221, the nut connector 275, the scale drum connector 230, and the scale drum 240, which will take the scale drum 240 back to the zero dose indicating position. The reset spring 295 thus serves to ensure that the scale drum 240 is not left in an intermediate position where an arbitrary dose is set.

When the lock nut 321 passes the transition point 288 the nut spring 296 expands and forces the protrusion 324 further proximally along the longitudinal track 289 while the primary nut 221 remains in position because the cap 204 is then fully mounted on the injection device 200. The nut assembly 220 is thereby immobilised on the spindle 205 and the reset spring 295 is securely cocked. Further, the axial displacement of the lock nut 321 causes the nut connector 275 to re-engage with the dose dial 255 and the set of teeth 226 to disengage from the set of teeth 278, thereby rendering the scale drum 240 accessible for displacement without impact on the nut assembly 220 and thus allowing for manual adjustment of the automatically set dose (compare FIG. 26 and FIG. 23).

The cap 204 and the loading rods 209 are designed such that when the cap 204 is fully mounted on the injection device 200 the nut assembly 220 has been moved to the dose prepared position and the injection device 200 is accordingly in a “DOSE PREPARED” state. This dose prepared position of the nut assembly 220 is maintained until the next dose expelling procedure is commenced, regardless of whether the position of the scale drum 240 is manually adjusted or not. The execution of the dose expelling procedure brings the nut assembly 220 from the dose prepared position to an end-of-dose position on the spindle 205 which uniquely corresponds to the movement of the scale drum 240 from its dose set position to the zero dose indicating position, and thereby to the particular dose expelled. When the scale drum 240 reaches the zero dose indicating position the injection device 200 is in a “DOSE DELIVERED” state.

The subsequent re-mounting of the cap 204 thus takes the nut assembly 220 back to the dose prepared position, regardless of from which end-of-dose position it departs, and due to the coupling between the nut assembly 220 and the scale drum 240 during the return movement of the nut assembly 220 the scale drum 240 is forced to undergo a movement which is the reverse of the one it underwent during the previous dose expelling procedure, and it is thereby returned to the exact same dose set position relative to the proximal housing part 201 it had when the nut assembly 220 last departed from the dose prepared position. So, the dose prepared position and the zero dose indicating position are both positions which are fixed with respect to the proximal housing part 201, whereas the end-of-dose position and the dose set position are both variable and depend on the specific user input.

1. A drug delivery device comprising:
a housing,
a dose setting mechanism operable to set a dose to be expelled from a variable volume reservoir, and
a dose delivery structure activatable during a dose expelling procedure to cause expelling of a set dose,
wherein the dose setting mechanism comprises:
a dose indicating structure for indicating a size of the set dose, the dose indicating structure being coupled with the dose delivery structure during the dose expelling procedure and moved relative to the housing to a zero dose indicating position, the zero dose indicating position being a position which is fixed with respect to the housing, and
a dose setting structure movable in a dose preparing direction relative to the housing to a dose prepared position to set a dose of a first size, the dose prepared position being a position along the dose preparing direction which is fixed with respect to the housing,
wherein the dose setting structure and the dose indicating structure are configured to undergo first correlated displacements relative to the housing during the dose expelling procedure and to undergo second correlated displacements relative to the housing during movement of the dose setting structure to the dose prepared position, the first correlated displacements and the second correlated displacements being mutually reverse, and
wherein when the dose setting structure is in the dose prepared position the dose indicating structure is selectively displaceable relative to the housing while the dose setting structure remains stationary in the dose preparing direction to allow adjustment of the dose of the first size and thereby setting of a dose of a second size.

2. A drug delivery device according to claim 1, wherein the housing comprises a window, and
wherein the dose indicating structure carries a plurality of dose indicia which are successively displayable through the window.

3. A drug delivery device according to claim 1, wherein the dose indicating structure is a scale drum, which scale drum is threadedly engaged with the housing.

4. A drug delivery device according to claim 1, wherein the dose setting structure and the dose indicating structure are arranged concentrically along a longitudinal axis of the drug delivery device, and the dose indicating structure surrounds at least a portion of the dose setting structure.

5. A drug delivery device according to claim 1, wherein the dose setting structure is configured to be moved to the dose prepared position in response to a predetermined user action.

6. A drug delivery device according to claim 5, further comprising:
a cap receiving portion adapted to receive and releasably retain a cap in a position where the cap covers a distal end portion of the drug delivery device,
wherein the cap receiving portion allows for an operative coupling between the cap and the dose setting structure, and
wherein the predetermined user action comprises mounting the cap onto the cap receiving portion.

7. A drug delivery device according to claim 6, wherein the cap comprises a dose arming structure configured for abutment with the dose setting structure.

8. A drug delivery device according to claim 4, wherein the dose delivery structure and the dose setting structure are arranged concentrically, and the dose setting structure surrounds at least a portion of the dose delivery structure.

9. A drug delivery device according to claim 1, wherein the dose indicating structure is selectively displaceable relative to the housing by operation of a dose adjustment structure, the dose adjustment structure being operable to displace the dose indicating structure only when the dose setting structure is in the dose prepared position.

10. A drug delivery device according to claim 4, further comprising:
a rotator connecting the dose setting structure and the dose indicating structure,
wherein the rotator comprises a longitudinally extending exterior track and the dose indicating structure comprises an interior protrusion for engagement with the exterior track,
wherein the rotator further comprises an interior projection and the dose setting structure comprises an exterior helical groove and an exterior longitudinal groove being connected with the exterior helical groove, and
wherein the interior projection is configured for engagement with the exterior longitudinal groove, when the dose setting structure is in the dose prepared position, and for non-self-locking threaded engagement with the exterior helical groove, when the dose setting structure is away from the dose prepared position.

11. A drug delivery device according to claim 4 or 10, further comprising:
a dose dial operable by a user to affect the position of the dose indicating structure relative to the housing when the dose setting structure is in the dose prepared position,
wherein when the dose setting structure is in the dose prepared position the dose setting structure and the dose indicating structure are rotationally interlocked, and the dose setting structure and the dose dial are rotationally interlocked.

12. A drug delivery device according to claim 11, further comprising:
a dose activation button for activating the dose delivery structure,
wherein the dose activation button is shiftable between a passive position in which the dose delivery structure is stationary and an activated position in which the dose delivery structure is activated and the dose indicating structure moves towards the zero dose indicating position, and
wherein the dose setting structure is configured to rotationally disengage from the dose dial in response to the dose activation button being shifted to the activated position and to remain rotationally disengaged from the dose dial until returned to the dose prepared position.

13. A drug delivery device according to claim 12, wherein the dose setting structure and the dose indicating structure are configured to undergo a purely axial relative motion in response to the dose activation button being shifted to the activated position, whereby the dose setting structure becomes rotationally locked with respect to the housing, and whereby relative rotation between the dose setting structure and the dose indicating structure becomes enabled.

14. A drug delivery device according to claim 13, wherein the dose setting structure comprises circumferentially distributed teeth enabling a rotational locking of the dose setting structure with respect to the housing in a number of angular positions which corresponds to the number of dose increments per revolution of the dose indicating structure.

15. A drug delivery device according to claim 1, further comprising:
a dose activation button, and
a pre-tensioned spring for delivering energy to activate the dose delivery structure in response to an operation of the dose activation button.
